(19) United States
(12) Patent Application Publication
(10) Pub. No.: US 2026/0327309 A1
(43) Pub. Date: Oct. 22, 2026

(54) COMPOSITIONS FOR TREATING CANCER AND METHODS OF USING THE SAME

(71) Applicant: BOARD OF REGENTS, THE UNIVERSITY OF TEXAS SYSTEM, Austin, TX (US)

(72) Inventor: Moran AMIT, Houston, TX (US)

(21) Appl. No.: 19/109,900

(22) PCT Filed: Sep. 8, 2023
(86) PCT No.: PCT/US2023/073793

§ 371 Date: Mar. 7, 2025

Related U.S. Application Data
(60) Provisional application No. 63/374,999, filed on Sep. 8, 2022.

(51) Int. Cl. C07K 16/28; A61K 39/00; C07K 16/24; G01N 33/575

(52) U.S. Cl. CPC C07K 16/2818; C07K 16/245; C07K 16/249; G01N 33/57557; A61K 2039/507; C07K 2317/21; C07K 2317/76

(57) ABSTRACT

The present disclosure relates to combination therapies comprising immune checkpoint inhibitors and antibodies directed to neurological biomarkers, compositions comprising immune checkpoint inhibitors and antibodies directed to neurological biomarkers, and methods of treatment using such combination therapies and/or compositions.

### CROSS REFERENCE TO RELATED APPLICATIONS

The present application claims the priority benefit of U.S. Provisional Application No. 63/374,999, filed Sep. 8, 2022, which is hereby incorporated by reference in its entirety.

### FIELD OF THE DISCLOSURE

The present disclosure relates to the fields of immunology, cancer therapy and neurology.

### BACKGROUND

Recent oncology research has focused on utilizing the immune system to attack tumor cells. For example, there have been remarkable advancements including immune checkpoint inhibitors, which have proven effective for treating a wide range of cancers.

Based on promising results from recent clinical trials, immune checkpoint inhibitors will likely become available for many other cancers. (see, e.g., Hamanishi et al., 2015; Ansell et al., 2015; McDermott et al., 2015; Early Breast Cancer Trials, 1998; Le et al., 2015).

Immunotherapy is so effective that it could benefit millions of cancer patients worldwide every year.

Cutaneous squamous cell carcinoma (cSCC) is the second most common form of skin cancer and more prevalent than melanoma within the United States. (see, e.g., Fania, L et al, Biomedicines 9(2):171 (2021)). cSCC is a cancer arising from malignant proliferation of the keratinocytes of the epidermis that has invaded into the dermis or beyond. cSCC is a deadly threat owing to its ability to metastasize to any organ in the body. Though most cases are treated by excision, a subset recur and become incurable via excision, with the number of deaths approximating melanoma (Karia et al, J. Am. Acad. Dermatol. 68(6): 957-66 (2013)). Those at high risk of recurrence may be eligible for adjuvant treatment options. Immunotherapies, such as immune checkpoint inhibitors, specifically anti-programmed death 1 (PD-1) antibodies, are a promising treatment option for those afflicted with cSCC.

However, subsets of the population afflicted cancer (e.g., cSCC) exhibit resistance to immunotherapies, specifically immune checkpoint inhibitors (e.g., an anti-PD-1 antibody therapy). Currently, not much is known regarding the association of neurological biomarkers related to degenerating tumor-associated nerves and their role in cancer and anticancer resistant responses. There is a need to identify relevant biomarkers to identify subjects likely to have anticancer resistant response to immunotherapies.

### DESCRIPTION OF FIGURES

FIG. 1A shows the waterfall plot of percentage change from baseline in target lesions per RECIST 1.1 and coded for the pathological complete response (PCR) and the major pathological response (MPR) following the cemiplimab (anti-PD-1 antibody) treatment.

FIG. 1B shows representative pretreatment (top left) and post-treatment (top right) photographs, coronal computed tomography (CT) images before treatment (middle left) and after treatment (middle right), and micrographs before treatment (bottom left) and after treatment (bottom right) of tumor specimens from a subject who achieved a major pathologic response (6-mm foci of residual viable tumor) following cemiplimab (anti-PD-1 antibody) treatment.

FIGS. 2A-2C show DAPI (4,6-diamidino-2-phenylindole) fluorescence microscopy staining of Creatine Kinase (CK) (FIG. 2A), Beta-III-Tubulin (FIG. 2B), Neurofilament Heavy chain (NfH) (FIG. 2C) at 500 μm.

FIGS. 3A-3B shows a dendrogram of potential biomarkers detailing cSCC responders and non-responders pre-treatment with cemiplimab (anti-PD-1 antibody) treatment.

FIGS. 4A-4B shows a dendrogram of potential biomarkers detailing cSCC responders and non-responders post-treatment with cemiplimab (anti-PD-1 antibody) treatment.

FIG. 5 shows the alpha diversity of the microbiome in a cSCC subject's tumor pre-and post-treatment.

FIG. 6A shows the study workflow as tumor samples from two anti-PD-1 neoadjuvant clinical trials for cutaneous squamous cell carcinoma were used for molecular analysis and validated using in vivo and in vitro models.

FIG. 6B shows clinical characteristics of patients from clinical trial cohorts. “R” designates responders to anti-PD-1 treatment. “NR” designates non-responders to anti-PD-1 treatment. “T” designates tumor. “N” designates nodal. “M” designates metastases.

FIG. 6C shows the peri-neural invasion rates in the clinical trial tumor samples according to response status.

FIG. 6D shows representative stains demonstrating expression of neural injury markers ATF3 and cJUN in tumor-associated nerves (TANs), with their distribution among neural (GFAP−) and Schwan (GFAP+) components.

FIG. 6E shows the histograms of mean SEM, ATF3, and cJUN expression levels.

FIG. 6F shows the gene set enrichment analysis (GSEA) of genes associated with TANs proximity to cancer cells.

FIG. 6G shows the denervation experiment design utilized on mice.

FIG. 6H shows the tumor growth plot (day 30) of the denervation experiment over 30 days.

FIG. 6I shows the nerve injury (axotomy) experiment design following tumor implantation.

FIG. 6J shows the tumor growth plot over 39 days of the axonotomy mouse experiment.

FIG. 7A shows a tumor volume bar plot of the denervated mice model at day 30.

FIG. 7B shows a tumor volume bar plot of the nerve injury experiment at day 39.

FIG. 7C shows an experiment design description of the nerve injury experiment in human leukocyte antigen (HLA) matched humanized huCD34+ NSG mice. FIG. 7D shows a tumor growth plot for the HLA matched humanized huCD34+ NSG mice in comparison to the control group over 42 days. FIG. 7E shows a tumor volume bar plot of the HLA matched humanized huCD34+ NSG mice, at day 40.

FIG. 8A shows the scanning electron microscopy (EM) image, a naïve dorsal root ganglia (DRG) neuron (left, inset, x50,000) with normal myelin sheath, is compared to a DRG neuron that was co-cultured with squamous cell carcinoma cells (SCC, round cells, right image) for 5 days.

FIG. 8B shows transmission electron microscopy (EM) images. Low-power field images are shown in the top row and high-power field images in the bottom row. The first column shows naïve neurons. The second column shows DRG neurons, which were co-cultured with SCC cells for 5 days. The third and fourth columns show the same images with the myelin and Schwann cells, respectively.

FIG. 8C shows electron microscopy (EM) images of the invasive cancer cells to the nerve inner layers, nerve filaments. The first column shows the scanning EM, and the second and third columns show the transmission EM.

FIGS. 8D-8E show multielectrode array recordings of normal skin (control) and cutaneous SCC showing similar baseline and reversion electrical activity with blunted evoked response in tumor specimens.

FIG. 8F shows the immunofluorescence (IF) stains of tumor samples from indented cutaneous SCC patient cohorts.

FIG. 8G shows a Pearson's correlation plot between markers of nerve damage and de-myelination.

FIG. 8H shows the transcriptional differences in DRG neuron that were co-cultured with IC8 SCC cells (DRG-IC8) compared to DRG neurons alone.

FIG. 8I shows a heatmap with the protein expression of neurodegeneration-associated markers enriched within intra-tumoral neural niches of non-responders patients compared with responders. Protein expression was measured by digital spatial profiling (DSP) and transformed into z-scores for representation.

FIG. 8J shows a heatmap demonstrating transcriptional differences between responders (R) and non-responders (NR), in neoadjuvant-treated tumor samples and the corresponding enriched pathways.

FIG. 9 is an immunohistochemistry stain against ERG marker for endothelial cells, and shows intact intratumoral vasculature (arrowheads), perineuroal inflammation (asterisks), and tumor-associated nerve damage (nerve marked with black arrowheads).

FIG. 10A shows a volcano plot which displays differential gene expression in DRG neurons co-cultured for 5 days, with human IC8 cutaneous squamous cell carcinoma (sSCC) cells versus DRG neurons co-cultured IC8 SCC cells and anti-PD-1.

FIG. 10B shows a heat map of differentially expressed genes between murine SKH primary TG neurons alone and TG neurons co-cultured with murine B6 cSCC.

FIGS. 10C-10D show volcano plots, which displays the differential gene expression in murine TG neurons versus TG neurons treated with anti-PD-1 (FIG. 10C) and TG neurons co-cultured with B6 cSCC cells without anti-PD-1 administration (FIG. 10D).

FIGS. 11A-11C show the nerve identification process utilized in digital spatial profiling (DSP) analysis. Neurofilament heavy responder chain (NFH) and β3-tubulin staining neoadjuvant-treated tumor samples of a responder (FIG. 11A) and non-responder (FIGS. 11B-11C) are shown. FIG. 11B shows perineural invasion via DPS. FIG. 11C shows intratumoral nerve invasion via DSP.

FIG. 11D shows the geometric region of interest (ROI) as a white rectangle, indicating a representative digital capture of the nerve (middle, shadowed, nerve capture panel, NCP) and the surrounding peri-neural niche; further assessed for immune markers (left, shadowed, immune capture panel, ICP).

FIG. 11E shows box plots with expression levels of neuro-protective markers (TMEM119 and ApoA-1) and marker of blood-nerve barrier reactivity and angiogenesis (CD31) in tumor-associated nerves (TANs) among baseline tumor samples.

FIG. 11F shows transcriptional differences between responders and non-responders to anti-PD-1 therapy in baseline tumor samples.

FIG. 12A shows unsupervised hierarchical clustering analysis (HCA) using single sample enrichment scores for cancer associated peripheral nerve degeneration (CAPND) related gene sets among patients.

FIG. 12B shows the perineural invasion (PNI) status among tumors from the Cancer Genome Atlas Program (TCGA) head and neck squamous cell carcinoma patients (HNSCC) patients based on their CAPND scores.

FIG. 12C shows the Kaplan-Meier analysis of overall survival (OS), progression-free interval (PFI), and disease-free interval (DFI) by CAPND scores group.

FIG. 12D shows the Kaplan-Meier estimates of overall survival (OS), progression-free interval (PFI), and disease-free interval (DFI) of the TCGA HNSCC patients stratified by the immune signature groups.

FIG. 13A shows the Kaplan-Meier analyses showing progression-free interval (PFI), disease-free interval (DFI), and overall survival (OS) plots for a head and neck mucosal squamous cell carcinoma (SCC) patient cohort from the TCGA database.

FIG. 13B shows a Bubble heatmap based on digital spatial profiling (DSP) protein matrix, exhibiting the correlation coefficients between immune and neural proteins expressed in the peri-neural niche of neoadjuvant-treated tumor samples of the clinical trial cohort.

FIG. 13C is a multiplex-immunofluorescence (IF) stain of healthy (NFH+B3T+ATF3−, top panels) and damaged (ATF3+cJUN+, bottom panels) tumor-associated nerves (TANs). The area around the nerves (i.e., peri-neural niche) was stained for CD163, CD8, LAG3 and PD1, Gramzyme B, and Perforin.

FIG. 13D is a bar plot representing IF-based cell density according to clinical response to anti-PD-1 therapy between responders and non-responders.

FIG. 13E shows the spatial transcriptomic analysis of tumor samples from an independent treatment naïve cutaneous SCC patient cohort.

FIG. 13F shows the spatial transcriptomic analysis of tumor samples from across the cohort independent treatment naïve cutaneous SCC patient cohort.

FIG. 13G shows the stacked bar plots representing sequenced tissue spots grouped according to the CAPND phenotype score.

FIG. 13H shows a bar plot and spatial analysis conducted on neoadjuvant-treated samples.

FIG. 14A shows eight tumor samples collected from huCD34+ NSG mice with and without nerve injury, for spatial transcriptomic analysis evaluating human and murine genes. Cancer associated peripheral nerve degeneration (CAPND), tumor-promoting inflammation, and anti-tumoral immunity are sorted from top to bottom, respectively.

Representative images and their respective sequenced spots are shown for tumors samples from a nerve-injured mice (D4) and a control mice (ND2) (top).

FIG. 14B is a ridge-plot which shows the frequency distribution of sequenced tissue spots (y-axis) at a given enrichment score (x-axis, right to the line) for each sample and each phenotype signature. Nerve injured animals (D1-4) and tumor-promoting inflammation phenotypes compared to animals without nerve injury (ND1-4).

FIG. 15A shows immunohistochemically (IHC) based cell count of the CD8+ cells in the clinical trial cohort, according to the clinical response status to anti-PD-1 therapy.

FIG. 15B shows IHC-based cell count of (i) PD-1+, (ii) PD-L1+ immune cells, and PD-L1+ tumor cells.

FIG. 15C shows Gene Ontology (GO) enrichment analyses of bulk tumor RNA sequencing of the different immune landscape in neoadjuvant-treated tumor sample of responders and non-responders.

FIG. 15D shows a Nanostring nCounter panCancer analysis of clinical trial tumor samples, including T regulatory cells (Tregs) and Tumor Growth Factor (TGF)-ß1 expression, based on response at base line and on treatment.

FIG. 15E shows a Nanostring nCounter PanCancer pathway enrichment analysis of neoadjuvant-treated samples according to response status.

FIG. 15F shows an exemplary schematic of a demyelination and inflammatory signal blockade mouse experiment.

FIG. 15G shows the tumor growth and viability plots of the different groups within the demyelinated mouse experiment.

### BRIEF SUMMARY

The present disclosure provides a combination therapy comprising (i) an antibody that specifically binds to an immune checkpoint protein (e.g., programmed death 1 (PD-1)), and (ii) an antibody directed to a neurological biomarker disclosed herein, as well as suitable delivery methods and compositions for the disclosed combination therapy.

Certain aspects of the disclosure provide a method of treating a subject suffering from cancer (e.g., cutaneous squamous cell carcinoma (cSCC)) comprising administering to the subject a therapeutically effective amount of a combination therapy comprising (i) an antibody that specifically binds to an immune checkpoint protein (e.g., programmed death 1 (PD-1)), and (ii) an antibody directed to a neurological biomarker disclosed herein.

Certain aspects of the disclosure provide a method of treating a subject suffering from cancer (e.g., cutaneous squamous cell carcinoma (cSCC)) comprising: (a) detecting or assessing a presence and/or absence of a neurological biomarker in a biological sample of the subject, wherein the neurological biomarker is selected from one or more of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription factor 3 (ATF3), and cJUN); (b) determining if the neurological biomarker detected or assessed in (a) is present and/or absent relative to a reference sample; and (c) administering to the subject a therapeutically effective amount of (i) an antibody that specifically binds to an immune checkpoint protein (e.g., programmed death 1 (PD-1)), and/or (ii) an antibody directed to a neurological biomarker disclosed herein if the neurological biomarker is detected and/or assessed to be present. In some aspects, the reference sample is a normal (i.e., non-cancerous) sample.

Certain aspects of the disclosure provide a method of treating a subject suffering from cancer (e.g., cutaneous squamous cell carcinoma (cSCC)) comprising: (a) detecting or assessing a presence and/or absence of a neurological biomarker in a biological sample of the subject, wherein the neurological biomarker is selected from one or more of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription factor 3 (ATF3) cJUN, Calcitonin Gene-Related Peptide (CGRP), Choline Acetyltransferase (ChAT), and Vesicular Acetylcholine Transporter (VAChT); (b) determining if the neurological biomarker detected or assessed in (a) is present and/or absent relative to a reference sample; and (c) administering to the subject a therapeutically effective amount of (i) an antibody that specifically binds to an immune checkpoint protein (e.g., programmed death 1 (PD-1)), and/or (ii) an antibody directed to a neurological biomarker disclosed herein if the neurological biomarker is detected and/or assessed to be present. In some aspects, the reference sample is a normal (i.e., non-cancerous) sample.

In some aspects, the combination therapy comprises: (i) an antibody that specifically binds to an immune checkpoint protein (e.g., programmed death 1 (PD-1)); and (ii) an antibody that specifically binds to a neurological biomarker selected from the group consisting of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription factor 3 (ATF3), cJUN, NEUROD1, RGPRD, TAC1, SSTR2, HAPLN4, SST, SMAD1, BHLE41, KLF7, and KLF6.

In some aspects, the combination therapy comprises: (i) an antibody that specifically binds to an immune checkpoint protein (e.g., programmed death 1 (PD-1)); and (ii) an antibody that specifically binds to a neurological biomarker selected from the group consisting of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription factor 3 (ATF3), cJUN, NEUROD1, RGPRD, TAC1, SSTR2, HAPLN4, SST, SMAD1, BHLE41, KLF7, KLF6, Calcitonin Gene-Related Peptide (CGRP), Choline Acetyltransferase (ChAT), and Vesicular Acetylcholine Transporter (VAChT).

In some aspects, the antibody that specifically binds to a neurological biomarker selected from the group consisting of NfH, NfL, TH, GFAP, OLIG2, CD39, ATF3, and cJUN.

In some aspects, the antibody that specifically binds to a neurological biomarker selected from the group consisting of NfH, NfL, TH, GFAP, OLIG2, CD39, ATF3, cJUN, CGRP, ChAT, and VAChT.

In some aspects, the neurological biomarker is NfH.

In some aspects, the neurological biomarker is NfL.

In some aspects, the neurological biomarker is TH.

In some aspects, the neurological biomarker is GFAP.

In some aspects, the neurological biomarker is OLIG2.

In some aspects, the neurological biomarker is CD39.

In some aspects, the neurological biomarker is ATF3.

In some aspects, the neurological biomarker is cJun.

In some aspects, the neurological biomarker is CGRP.

In some aspects, the neurological biomarker is ChAT.

In some aspects, the neurological biomarker is VAChT.

In some aspects, the antibody is selected from the group consisting of an anti-PD-1 antibody, anti-CTLA-4 antibody, anti-LAG-3 antibody, anti-4-1BB antibody, anti-OX-40 antibody, anti-CD27 antibody, anti-CD28 antibody, anti-TIM3 antibody, anti-B7-H3 antibody, VISTA antibody, anti-IDO-1 antibody, and any combination thereof.

In some aspects, the antibody is an anti-PD-1 antibody.

In some aspects, the anti-PD-1 antibody is selected from the group consisting of nivolumab, pembrolizumab, cemiplimab, and dostarlimab.

In some aspects, the immune checkpoint inhibitor, e.g., anti-PD-1 antibody, and the antibody directed to a neurological biomarker are in the same composition.

In some aspects, the immune checkpoint inhibitor, e.g., anti-PD-1 antibody, and the antibody directed to a neurological biomarker are in separate compositions.

In some aspects, the immune checkpoint inhibitor, e.g., anti-PD-1 antibody, is suitable for intratumorally, intravenously, intraperitoneally, intramuscularly, intrathecally or subcutaneous delivery.

In some aspects, the immune checkpoint inhibitor, e.g., anti-PD-1 antibody, is suitable for intravenous delivery.

In some aspects, the antibody directed to a neurological biomarker is suitable for intratumorally, intravenously, intraperitoneally, intramuscularly, intrathecally or subcutaneous delivery.

In some aspects, the combination therapy disclosed herein comprising the antibody that specifically binds to an immune checkpoint protein, e.g, anti-PD-1 antibody, and the antibody directed to a neurological biomarker is suitable for intratumorally, intravenously, intraperitoneally, intramuscularly, intrathecally or subcutaneously delivery in the same composition.

Certain aspects of the disclosure are directed to a method of treating a subject suffering from cancer (e.g., cutaneous squamous cell carcinoma (cSCC)) comprising administering to the subject a therapeutically effective amount of a combination therapy disclosed herein to the subject.

In some aspects, the immune checkpoint inhibitor (e.g., anti-PD-1 antibody) and the antibody directed to a neurological biomarker are administered simultaneously or sequentially.

In some aspects, the immune checkpoint inhibitor (e.g., anti-PD-1 antibody) and the antibody directed to a neurological biomarker are administered in the same composition.

In some aspects, the immune checkpoint inhibitor (e.g., anti-PD-1 antibody) and the antibody directed to a neurological biomarker are administered in different compositions.

In some aspects, the immune checkpoint inhibitor (e.g., anti-PD-1 antibody) is administered prior to or at the time of administration of the antibody directed to a neurological biomarker.

In some aspects, the biological sample is selected from a group consisting of blood, urine, tumor tissue, and cerebrospinal fluid (CSF).

In some aspects, if the subject is identified as exhibiting undetectable expression of or absence of expression of a neurological biomarker (e.g., in a biological sample), the subject is not administered (i) an antibody that specifically binds to an immune checkpoint protein (e.g., programmed death 1 (PD-1)), and/or (ii) an antibody directed to a neurological biomarker disclosed herein.

In some aspects, if the subject is identified as exhibiting an expression of or detecting the presence of a neurological biomarker (e.g., in a biological sample), the subject is administered (i) an antibody that specifically binds to immune checkpoint protein (e.g., programmed death 1 (PD-1)), and/or (ii) an antibody directed to a neurological biomarker disclosed herein.

In some aspects, the administration of the immune checkpoint inhibitor (e.g., anti-PD-1 antibody) inhibits the recurrence of a lesion (e.g., a tumor).

In some aspects, the cancer is selected from the group consisting of breast cancer, bladder cancer, cervical cancer, colon cancer, head and neck cancer, Hodgkin lymphoma, liver cancer, lung cancer, renal cell cancer (a type of kidney cancer), skin cancer (including melanoma, squamous cell carcinoma, or a cutaneous squamous cell carcinoma (cSCC)), stomach cancer, rectal cancer, or any combination thereof.

In some aspects, the cSCC is selected from the group consisting of advanced cSCC, metastatic cSCC, locally advanced cSCC, resectable cSCC, unresectable cSCC, recurrent cSCC, and any combination thereof.

### DETAILED DESCRIPTION

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Systéme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein, the term “biomarker” or “neurological biomarker” includes detectable molecules such as antibodies, genes, DNA, RNA, miRNA, fragments of RNA, fragments of DNA, peptides, proteins, lipids, or other biological material whose presence, absence, level or activity is correlative of or predictive of a condition, a predisposition, toxicity, damage, or disease. A biomarker can be a peptide or a protein. In some aspects, detection of the presence or absence of protein, or increases or decreases in protein levels correlates with the presence or absence of a condition or predisposition (e.g., to responsiveness to treatment or to a disease). As used herein, “peptide” means peptides of any length and includes proteins. The terms “polypeptide” and “oligopeptide” are used herein without any particular intended size limitation, unless a particular size is otherwise stated.

The term “tumor associated nerve” or “TAN”, as used herein refers to peripheral nerves fibers found within or in close proximity to tumors.

The term “peripheral nerve invasion” or “PNI”, as used herein refers a histo-morphological phenomenon. PNI is understood as the presence of tumor cells abutting or in close proximity to a nerve with encirclement of at least a third of the nerve circumference by tumor; or the presence of cancer cells within the epineurial, perineurial, and/or endoneurial compartments of a nerve.

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition (e.g., formation of a hydrogel which may then be optionally incorporated into a patch). Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

The term “effective amount” or “pharmaceutically effective amount” or “therapeutically effective amount” as used herein refers to the amount or quantity of a drug or combination therapy or composition or pharmaceutically active substance which is sufficient to elicit the required or desired therapeutic response, or in other words, the amount which is sufficient to elicit an appreciable biological response when administered to a subject.

The term “unit dosage form” or “unit dose composition” as used herein refers to a device containing a quantity of the therapeutic compound, said quantity being such that one or more predetermined units may be provided as a single therapeutic administration.

The term “diagnostic amount” of a marker refers to an amount or detectable level (e.g., indicating absence, presence, or expression level) of a marker in a subject's sample. A diagnostic amount can be either in absolute amount (e.g., μg/ml) or a relative amount (e.g., relative intensity of signals). In some aspects, the diagnostic amount is set to determine the presence or absence of the marker in the sample.

A “control amount” of a marker can be any amount or a range of amount which is to be compared against a test amount of a marker. A control amount can be either in absolute amount (e.g., ug/ml) or a relative amount (e.g., relative intensity of signals).

The terms “treatment”, “treating”, “treat” and the like are used herein to generally refer to obtaining a desired pharmacologic and/or physiologic effect. The effect may be prophylactic in terms of completely or partially preventing or reducing the risk of developing or worsening of a disease or symptom thereof and/or may be therapeutic in terms of a partial or complete stabilization or cure for a disease and/or adverse effect attributable to the disease.

In some aspects, treatment of a disease (e.g., cancer) in a mammal, particularly a human, can include: (a) preventing the disease or symptom from occurring in a subject which may be predisposed to the disease or symptom but has not yet been diagnosed as having it; (b) inhibiting the disease symptom, i.e., arresting its development; or (c) reducing or relieving the disease symptom, i.e., causing regression of the disease (e.g., cancer) or symptom.

The term “cancer” includes solid tumors, as well as, hematologic tumors and/or malignancies. A “precancer cell” or “precancerous cell” is a cell manifesting a cell proliferative disorder that is a precancer or a precancerous condition.

A “cancer” refers a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream.

A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate markers.

The cancer cells can be breast cancer cells, breast cancer cells, bladder cancer cells, cervical cancer cells, colon cancer cells, head and neck cancer cells, Hodgkin lymphoma cells, liver cancer cells, lung cancer cells, renal cell cancer (a type of kidney cancer) cells, skin cancer cells (including melanoma cells, squamous cell carcinoma cells, or a cutaneous squamous cell carcinoma cells (cSCC)), stomach cancer cells, and rectal cancer cells.

As used herein, “treating cancer”, “cancer treatment” and the like are not intended to be an absolute terms. In some aspects, the compositions and methods of the disclosure seek to reduce the size of a tumor or number of cancer cells, cause a cancer to go into remission, or prevent growth in size or cell number of cancer cells. In some circumstances, treatment with the leads to an improved prognosis.

The term “cell proliferative disorder” refers to conditions in which unregulated or abnormal growth, or both, of cells can lead to the development of an unwanted condition or disease, which may or may not be cancerous. Exemplary cell proliferative disorders of the application encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. Preferably, the methods provided herein are used to treat a symptom of cancer.

The term “therapeutic agent” as used herein refers to a compound or molecule that is useful in the treatment of a disease (e.g., cancer). In some aspects, a therapeutic agent can be administered separately, concurrently or sequentially with another therapeutic agent (e.g., an antibody moiety).

The term an “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

Antibodies typically bind specifically to their cognate antigen with high affinity, reflected by a dissociation constant (KD) of 10−5 to 10−11 M−1 or less. Any KD greater than about 10−4 M−1 is generally considered to indicate nonspecific binding. As used herein, an Ab that “binds specifically” to an antigen refers to an Ab that binds to the antigen and substantially identical antigens with high affinity, which means having a KD of 10−7 M or less, preferably 10−8 M or less, even more preferably 5×10−9 M or less, and most preferably between 10−8 M and 10−10 M or less, but does not bind with high affinity to unrelated antigens. An antigen is “substantially identical” to a given antigen if it exhibits a high degree of sequence identity to the given antigen, for example, if it exhibits at least 80%, at least 90%, preferably at least 95%, more preferably at least 97%, or even more preferably at least 99% sequence identity to the sequence of the given antigen.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

The term an “isolated antibody” refers to an Ab that is substantially free of other Abs having different antigenic specificities (e.g., an isolated Ab that binds specifically to PD-1 is substantially free of Abs that bind specifically to antigens other than PD-1). An isolated Ab that binds specifically to PD-1 may, however, have cross-reactivity to other antigens, such as PD-1 molecules from different species. Moreover, an isolated Ab may be substantially free of other cellular material and/or chemicals.

The term “monoclonal antibody” (“mAb”) refers to a preparation of Ab molecules of single molecular composition, i.e., Ab molecules whose primary sequences are essentially identical, and which exhibits a single binding specificity and affinity for a particular epitope. A mAb is an example of an isolated Ab. MAbs may be produced by hybridoma, recombinant, transgenic or other techniques known to those skilled in the art.

The term a “human” antibody (HuMAb) refers to an Ab having variable regions in which both the framework and CDR regions are derived from human germline immunoglobulin sequences. Furthermore, if the Ab contains a constant region, the constant region also is derived from human germline immunoglobulin sequences. The human Abs of the disclosure may include amino acid residues not encoded by human germline immunoglobulin sequences (e.g., mutations introduced by random or site-specific mutagenesis in vitro or by somatic mutation in vivo). However, the term “human antibody,” as used herein, is not intended to include Abs in which CDR sequences derived from the germline of another mammalian species, such as a mouse, have been grafted onto human framework sequences. The terms “human” Abs and “fully human” Abs and are used synonymously. The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

The term a “humanized” antibody refers to an Ab in which some, most or all of the amino acids outside the CDR domains of a non-human Ab are replaced with corresponding amino acids derived from human immunoglobulins. In one aspect, of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non contiguous regions of a polypeptide or polypeptides (conformational, non-linear, discontinuous, or non-contiguous epitope). In certain aspects, the epitope to which an antibody or antigen-binding fragment thereof binds can be determined by, e.g, NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g, site-directed mutagenesis mapping). For X-ray crystallography, crystallization may be accomplished using any of the known methods in the art (e.g, Giege R et al, (1994) Acta Crystallogr D Biol Crystallogr 50(Pt 4): 339-350; McPherson A (1990) Eur J Biochem 189:1-23; Chayen N E (1997) Structure 5:1269-1274; McPherson A (1976) J Biol Chem 251: 6300-6303). Antibody/antigen-binding fragment thereof: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff HW et al., ; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter CW; Roversi P et al, (2000) Acta Crystallogr D Biol Crystallogr 56(Pt 10): 1316-1323). Mutagenesis mapping studies can be accomplished using any method known to one of skill in the art. See, e.g, Champe M et al, (1995) J Biol Chem 270:1388-1394 and Cunningham B C & Wells J A (1989) Science 244:1081-1085 for a description of mutagenesis techniques, including alanine scanning mutagenesis techniques.

The term a polypeptide, antibody, polynucleotide, vector, cell, or composition, which is “isolated”, is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in certain aspects, the polypeptides can occur as single chains or associated chains.

The term “pharmaceutical formulation” or “pharmaceutical composition” refers to a preparation which is in such form as to permit the biological activity of the active ingredient to be effective, and which contains no additional components which are unacceptably toxic to a subject to which the formulation would be administered. The pharmaceutical formulation can be sterile.

The term “administering” refers to the physical introduction of a composition comprising a therapeutic agent to a subject, using any of the various methods and delivery systems known to those skilled in the art. Preferred routes of administration for antibodies of the include intratumoral, intramuscular, intrathecal, intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Alternatively, an antibody of the disclosure can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The terms “subject” and “patient” are used interchangeably. The subject can be an animal. In some aspects, the subject is a mammal such as a non-human animal (e.g, cow, pig, horse, cat, dog, rat, mouse, monkey or other primate, etc.). In some aspects, the subject is a human.

The term “a subject in need of treatment” or “treating a subject suffering from” refers to an individual or subject that has been diagnosed with a disease or disorder, e.g., a cancer or a cell proliferative disorder.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease.

As used herein, “refractory” as used herein refers to a disease, such as cancer, which does not respond to treatment. In an aspect, a refractory cancer may be resistant to treatment before or at the beginning of the treatment. In other aspect, a refractory cancer may become resistant during treatment. Refractory cancers are also called resistant cancers. In some aspects, refractory or recurrent malignant tumors can use the treatment methods disclosed herein.

As used herein, “relapsed” as used herein refers to the return of the signs and symptoms of a disease (e.g. cancer) or the return of a disease such as cancer during a period of improvement, for example, after a therapy, such as a previous treatment of cancer therapy.

The term “serum concentration” generally refers to the amount of a drug or other compound in the circulation, both bound to proteins and unbound, the latter of which generally corresponds to the therapeutically active fraction.

The term “bioavailability” generally refers to the rate and extent to which the active ingredient is absorbed from a drug product and becomes available at the site of action.

“Bioequivalence” is a term in pharmacokinetics generally used to assess the expected in vivo biological equivalence of two proprietary preparations of a drug. Two pharmaceutical products are bioequivalent if they are pharmaceutically equivalent and their bioavailabilities (rate and extent of availability) after administration in the same molar dose are similar to such a degree that their effects, with respect to both efficacy and safety, can be expected to be essentially the same.

As used herein, the expression “a subject in need thereof” means a human or non-human mammal that exhibits one or more symptoms or indications of skin cancer, and/or who has been diagnosed with skin cancer, including a solid tumor and who needs treatment for the same. In many aspects, the terms “subject” and “patient” are used interchangeably. The expression includes subjects with primary, established, or recurrent cancers. In specific aspects, the expression includes human subjects that have and/or need treatment for cancer.

The expression also includes subjects with primary or metastatic tumors (advanced malignancies). In certain aspects, the expression includes patients with a solid tumor that is resistant to or refractory to or is inadequately controlled by prior therapy (e.g., surgery or treatment with an anti-cancer agent such as carboplatin or docetaxel). In certain aspects, the expression includes patients with a tumor lesion that has been treated with one or more lines of prior therapy (e.g., surgically removed), but which has subsequently recurred. In certain aspects, the expression includes subjects with a skin cancer tumor lesion who are not candidates for curative surgery or curative radiation, or for whom conventional anti-cancer therapy is inadvisable, for example, due to toxic side effects. In other aspects, the expression includes subjects with a skin cancer tumor lesion for which surgical removal is planned. In other aspects, the expression includes subjects for whom the risk of recurrence is high due to prior history of recurrence after surgery.

It is to be understood that headers are provided solely for ease of reading, and are not intended to be limiting. Aspects disclosed under one or more headers can be applicable to or combinable with aspects disclosed under one or more other headers.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### II. Neurological Biomarkers

Certain aspects of the disclosure relate to the use of neurological biomarkers in assessing a cancer or tumor's response to neoadjuvant immune checkpoint protein (e.g. PD-1) blockade independent of other commonly studied immunotherapy response drivers such as inflammation, tumor mutational burden, and the microbiome. In some aspects, the neurological biomarkers disclosed herein (e.g. neurofilament light chain) can function as a prognostic to determine the estimated efficacy of a checkpoint inhibitor treatment (e.g. PD-1 blockade) for cancer (e.g. cSCC). In some aspects, the neurological biomarkers disclosed herein (e.g. neurofilament light chain) can be targeted as a complement therapy to current cancer therapies involving checkpoint inhibitors (e.g., anti-PD-1 antibodies such as cemiplimab), thus improving the therapeutic response.

In some aspects, the neurological biomarker disclosed herein is optionally a polynucleic acid such as an oligonucleotide.

In some aspects, the neurological biomarkers are optionally analyzed in combinations of multiple biomarkers in the same sample, samples taken from the same subject at the same or different times, or in a sample from a subject and another sample from another subject or a control subject.

A neurological biomarker can include a biomarker that is associated with, affected by, activated by, effects, or otherwise associates with a neuronal cell or nerve damage.

Examples of the neurological biomarker of the disclosure includes one or more of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription Factor 3(ATF3), cJUN, NEURODI, RGPRD, TAC1, SSTR2, HAPLN4, SST, SMAD1, BHLE41, KLF7, or KLF6.

A neurological biomarker can include a biomarker that is associated with, affected by, activated by, effects, or otherwise associates with a neuronal cell or nerve damage. Examples of the neurological biomarker of the disclosure includes one or more of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription Factor 3(ATF3), cJUN, NEUROD 1, RGPRD, TAC1, SSTR2, HAPLN4, SST, SMAD1, BHLE41, KLF7, KLF6, Calcitonin Gene-Related Peptide (CGRP), Choline Acetyltransferase (ChAT), or Vesicular Acetylcholine Transporter (VAChT).

In some aspects, the neurological biomarker selected from the group consisting of NfH, NfL, TH, GFAP, OLIG2, CD39, ATF3, cJUN, and any combination thereof.

In some aspects, the neurological biomarker selected from the group consisting of NfH, NfL, TH, GFAP, OLIG2, CD39, ATF3, cJUN, CGRP, ChAT, VAChT, and any combination thereof.

In some aspects, the neurological biomarkers are selected from NEFL, NEFH, NEFM, NEUROD1, MRGPRD, TAC1, SSTR2, HAPLN4, SST, and any combination thereof.

In some aspects, the neurological biomarkers are selected from ATF3, JUN, SOX1, SMAD1, BHLHE41, KLF7, KLF6, and any combination thereof.

In some aspects, the neurological biomarkers are selected from CD8A, GZMB, PRF1, CD4, IL2, CD86, IRF8, TNF, PSMB10, HLADQA1, HLADRA, HLADRB1, and any combination thereof.

In some aspects, the neurological biomarkers are selected from CD204, CD206, CD163, CD68, IL-10, CD4, FOXP3, and any combination thereof.

In some aspects, the neurological biomarkers are selected from CGRP, ChAT, VAChT, and any combination thereof.

Neurofilaments are the major cytoskeletal component of nerve cells. It is important to maintain the axon's caliber and morphology intact, affecting the rate and accuracy of nerve transmission. There are three different neurofilament chains, defined by their size, neurofilament light chain (NF-L), neurofilament medium chain (NF-M) and neurofilament heavy chain (NF-H), respectively. The neurofilament light chain constitutes the backbone of the heavy chain, which aggregates to form neurofilament fibers. NF-L is the first expressed substructure in the neurofilament protein, the molecular weight is 68KD, NF-H is the last expressed, the molecular weight is 205 KD, and the two expressions are independent. Axonal proteins can be detected quantitatively because damaged nerve cell contents are released to peripheral compartments. In some aspects, the neurological biomarker is NfH. In some aspects, the neurological biomarker is NfL.

Tyrosine hydroxylase (TH) is a marker for dopamine, norepinephrine, and epinephrine-containing (catecholamine) neurons and endocrine cells. Tyrosine hydroxylase TH is a necessary enzyme to create neurotransmitters and protect the body against oxidative stress. Tyrosine hydroxylase is mainly present in the cytosol, although it also is found in some extent in the plasma membrane of the cell. Abnormal levels of TH expression are associated with neurodegenerative diseases and neuronal damage, and is used as a neurological biomarker which can be found in biological samples. In some aspects, the neurological biomarker is TH.

Glial fibrillary acidic protein (GFAP) is an intermediate filament protein found in astrocytes in the brain. GFAP is understood to be involved in the structure of the cell's cytoskeleton and to maintain astrocyte integrity. Damaged astrocytes release GFAP into cerebrospinal fluid and blood. This protein is not routinely secreted in blood and is only released after cell death or injury. GFAP is a key neurological biomarker in neuronal damage. In some aspects, the neurological biomarker is GFAP.

Oligodendrocyte transcription factor (OLIG2) is a basic helix-loop transcription factor encoded by the Olig2 gene. The protein weights 32 kDa in size and is expressed and located in the central nervous system. OLIG2 is mostly expressed in restricted domains of the brain and spinal cord ventricular zone which give rise to oligodendrocytes and specific types of neurons. OLIG2 first directs motor neuron fate by establishing a ventral domain of motor neuron progenitors and promoting neuronal differentiation. OLIG2 then switches to promoting the formation of oligodendrocyte precursors and oligodendrocyte differentiation at later stages of development. It is known for determining motor neuron and oligodendrocyte differentiation. In some aspects, the neurological biomarker is OLIG2.

Ectonucleoside triphosphate diphosphohydrolase-1 (ENTPD1) is also denoted as a protein, labelled “CD39”. CD39 is an ectoenzyme that hydrolases adenosine triphosphate/uridine diphosphate and adenosine diphosphate/uridine diphosphate to the respective nucleosides such as adenosine monophosphate. CD39 is located and expressed in the the central nervous system. It is present in negligible concentrations under normal conditions (10-100 nM), extracellular ATP rapidly increases in response to tissue injury and hypoxia11 and can be found at high concentrations in tumors (1-50 μM), thus, CD39 is a known biomarker in the field of neurology. In some aspects, the neurological biomarker is CD39.

Activating Transcription Factor 3 (ATF3) is a stress-induced transcription factor that plays vital roles in modulating metabolism, immunity, and oncogenesis. ATF3 is induced by a variety of signals, including many of those encountered by cancer cells, and is involved in the complex process of cellular stress response. ATF3 is a known biomarker in the field of neurology. In some aspects, the neurological biomarker is ATF3.

c-Jun or cJUN, is a common transcription factor, and a major component of the dimeric transcription factor activator protein-1, which is essential for transcriptional response to extracellular signaling. c-Jun can immediately express and generate transcription factors, regulate the transcription and expression of other genes, affect the growth, development and differentiation of normal cells, and lead to malignant transformation of cells. In some aspects, the neurological biomarker is cJUN.

Calcitonin gene related peptide (CGRP) is a member of the calcitonin family of peptides. Calcitonin is mainly produced by thyroid C cells whilst CGRP is secreted and stored in the nervous system. It is produced in the peripheral and central neurons. It acts as a potent peptide vasodilator and functions in the transmission of nociception. In some aspects, the neurological biomarker is CGRP.

Choline Acetyltransferase (ChAT) or (CAT), is present in both the central nervous system, and peripheral nerous system. It is a transferase enzyme responsible for the synthesis of acetylcholine. Concentration of ChAT is found highest in cholingeric neurons. In some aspects, the neurological biomarker is ChAT.

Vesicular acetylcholine transporter (VAChT), is a neurotransmitter transporter which loads acetylcholine into secretory organelles in neurons. VAChT is expressed selectively in all known cholinergic neurons. In some aspects, the neurological biomarker is VAChT.

Neurological biomarkers can correspond to their respective amino acid structures found under their respective GENBANK® Accession No. NfL can be correspond to GENBANK® Accession No. NP_006149. NfH can correspond to GENBANK® Accession No. NP_066554.2. TH can correspond to GENBANK® Accession Nos. NP_000351, NP_954986, NP_954987, NP_954986.2, and NP_954987.2. GFAP can correspond to GENBANK® Accession Nos.: NP_001124491, NP_001229305 NP_002046, and NP_001350775. OLIG2 can correspond to GENBANK ® Accession No. NP_005797. CD39 can correspond to GENBANK® Accession Nos.: NP_001091645, NP_001157650, NP_001157651, NP_001157653, and NP_001157654. ATF3 can correspond to GENBANK® Accession Nos. NP_001025458, NP_001035709, NP_001193413, NP_001193415, and NP_001193417. cJUN can correspond to GENBANK® Accession No. NP_002219. VACHT can correspond to GENBANK® Accession No. NP_003046.

In some aspects, the neurological biomarker comprises a plurality of neurological biomarkers selected from the group consisting of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription Factor 3 (ATF3), or cJUN.

In some aspects, the neurological biomarker comprises a plurality of neurological biomarkers selected from the group consisting of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription Factor 3 (ATF3), cJUN, Calcitonin Gene-Related Peptide (CGRP), Choline Acetyltransferase (ChAT), or Vesicular Acetylcholine Transporter (VAChT).

In some aspects, the neurological biomarker comprises a plurality of neurological biomarkers selected from the group consisting of NfH, NfL, TH, GFAP, OLIG2, CD39, ATF3, or cJUN.

In some aspects, the neurological biomarker comprises a plurality of neurological biomarkers selected from the group consisting of NfH, NfL, TH, GFAP, OLIG2, CD39, ATF3, cJUN, CGRP, ChAT, and VAChT.

In some aspects, the plurality of neurological biomarkers are measured in separate samples. It is appreciated that some biomarkers are optionally measured in the same sample while other biomarkers are measured in other samples. Illustratively, some biomarkers are optionally measured in serum while the same or other biomarkers are measured in CSF, blood, urine, tumor tissue, or other biological sample.

In some aspects, biomarker analysis or assessment can be performed using biological samples or fluids. In some aspects, the biological samples can include, cells, tissues, tumor tissues, cerebral spinal fluid (CSF), artificial CSF, whole blood, serum, plasma, cytosolic fluid, urine, feces, stomach fluids, digestive fluids, saliva, nasal or other airway fluid, vaginal fluids, semen, buffered saline, saline, water, or other biological fluid recognized in the art.

It is appreciated that neurological biomarkers, in addition to being obtained from CSF and serum, are also illustratively readily obtained from whole blood, plasma, saliva, urine, as well as solid tissue biopsy. In some aspects, CSF is a preferred sampling fluid owing to direct contact with the nervous system. In some aspects, other biological fluids have advantages in being sampled such as blood, plasma, serum, saliva or urine.

In some aspects, the biological sample is obtained from a subject by conventional techniques. For example, CSF can be obtained by lumbar puncture. Blood can be obtained by venipuncture, while plasma and serum can be obtained by fractionating whole blood according to known methods. A biological sample (e.g., urine sample) can be obtained from a patient and stored for later processing and analysis.

In some aspects, the biological marker is assessed from a solid tissue sample. Surgical techniques for obtaining solid tissue samples are well known in the art. For example, methods for obtaining a nervous system tissue sample are described in standard neurosurgery texts such as Atlas of Neurosurgery: Basic Approaches to Cranial and Vascular Procedures, by F. Meyer, Churchill Livingstone, 1999; Stereotactic and Image Directed Surgery of Brain Tumors, 1st ed., by David G. T. Thomas, WB Saunders Co., 1993; and Cranial Microsurgery: Approaches and Techniques, by L. N. Sekhar and E. De Oliveira, 1st ed., Thieme Medical Publishing, 1999. Methods for obtaining and analyzing brain tissue are also described in (Belay et al., Arch. Neurol. 58:1673-1678 (2001); and Seijo et al., J. Clin. Microbiol. 38:3892-3895 (2000)).

An exemplary process for detecting the presence or absence of one or more neurological biomarkers in a biological sample involves obtaining a biological sample from a subject, such as a human, contacting the biological sample with an agent capable of detecting of the marker being analyzed, illustratively including an antibody or aptamer, and analyzing binding of the agent optionally after washing. Those samples having specifically bound agent express the marker being analyzed.

In some aspects, a process for assessing whether a subject suffering from a cancer or tumor (e.g., cSCC) will be administered a therapeutic agent disclosed herein is provided, which comprises detecting the presence and/or absence and/or measuring the quantity of a first neurological biomarker in a sample from the subject.

An exemplary process disclosed herein can be used to detect one or more neurological biomarkers disclosed herein in a biological sample. The quantity of expression of one or more other neurological biomarkers in a sample can be compared with appropriate controls such as a first sample known to express detectable levels of the marker being analyzed (positive control) and a second sample known to not express detectable levels of the marker being analyzed (a negative control). For example, in vitro techniques for detection of a marker can include enzyme linked immunosorbent assays (ELISAs), western blots, immunoprecipitation, and immunofluorescence. Also, in vivo techniques for detection of a marker can include introducing a labeled agent that specifically binds the marker into a biological sample or test subject. For example, the agent can be labeled with a radioactive marker whose presence and location in a biological sample or test subject can be detected by standard imaging techniques.

In some aspects, baseline levels of biomarkers include those levels obtained in the target biological sample in a subject in the absence of a known condition. These levels need not be expressed in hard concentrations, but may instead be known from parallel control experiments and expressed in terms of fluorescent units, density units, and the like. Methods for determining the concentration of baseline levels of biomarkers is within the skill of the art. In some aspects, the baseline levels comprise the quantity or activity of a biomarker in a sample from one or more subjects that are not suspected of having a condition associated with the biomarker.

In some aspects, a biological sample is assayed by mechanisms known in the art for detecting or identifying the presence of one or more biomarkers present in the biological sample. Based on the amount or presence of a target biomarker in a biological sample, a ratio of one or more biomarkers is optionally calculated. The ratio is optionally the level of one or more biomarkers relative to the level of another biomarker in the same or a parallel sample, or the ratio of the quantity of the biomarker to a measured or previously established baseline level of the same biomarker in a subject known to be free of a pathological condition. In some aspects, the assessment of the level of neurological biomarker in a subject is used to determine whether or a not a subject (e.g., suffering from cSCC) will benefit from an immune checkpoint therapy, such as anti-PD-1 antibody.

As used herein a “level” is either a positive ratio wherein the level of the target is greater than the target in a second sample or relative to a known or recognized baseline level of the same target. A negative ratio describes the level of the “level” as lower than the target in a second sample or relative to a known or recognized baseline level of the same target. A neutral ratio describes no observed change in a target biomarker.

### Iii. Method of Identifying a Cancer Subject for Immunotherapy

As described herein, biomarkers can be used to determine whether to treat or not treat a subject suffering from cancer or a tumor (e.g., cSCC). For example, the disclosure provides methods for treating or not treating a subject suffering from cancer (e.g., cSCC) with a checkpoint inhibitor therapy (e.g., an anti-PD-1 antibody) based on, e.g., the presence, absence, or concentration (relative or absolute) of a neurological biomarker, or combination of neurological biomarkers described herein.

The presence and/or absence, detectable level or expression profile of a neurological biomarker can be utilized to determine the method for treating a subject suffering from a cancer or a tumor (e.g., cSCC). In some aspects, this treatment can be a combination therapy and/or immune checkpoint inhibitor (e.g., an anti-PD-1) therapy.

In some aspects, the neurological biomarker is selected from one or more of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription Factor 3 (ATF3), and cJUN.

In some aspects, the neurological biomarker is selected from one or more of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription Factor 3 (ATF3), cJUN, Calcitonin Gene-Related Peptide (CGRP), Choline Acetyltransferase (ChAT), and Vesicular Acetylcholine Transporter (VAChT).

In some aspects, the neurological biomarker comprises one or more NfH, NfL, TH, GFAP, OLIG2, CD39, ATF3, or cJUN.

In some aspects, the neurological biomarker comprises one or more NfH, NfL, TH, GFAP, OLIG2, CD39, ATF3, cJUN, CGRP, ChAT, and VAChT.

In some aspects, the neurological biomarker is NfH.

In some aspects, the neurological biomarker is NfL.

In some aspects, the neurological biomarker is TH.

In some aspects, the neurological biomarker is GFAP.

In some aspects, the neurological biomarker is OLIG2.

In some aspects, the neurological biomarker is CD39.

In some aspects, the neurological biomarker is ATF3.

In some aspects, the neurological biomarker is cJUN.

In some aspects, the neurological biomarker is CGRP.

In some aspects, the neurological biomarker is ChAT.

In some aspects, the neurological biomarker is VAChT.

In some aspects, the neurological biomarkers are selected from NEFL, NEFH, NEFM, NEUROD1, MRGPRD, TAC1, SSTR2, HAPLN4, SST, and any combination thereof.

In some aspects, the neurological biomarkers are selected from ATF3, JUN, SOX1, SMAD1, BHLHE41, KLF7, KLF6, and any combination thereof.

In some aspects, the neurological biomarkers are selected from CD8A, GZMB, PRF1, CD4, IL2, CD86, IRF8, TNF, PSMB10, HLADQA1, HLADRA, HLADRB1, and any combination thereof.

In some aspects, the neurological biomarkers are selected from CD204, CD206, CD163, CD68, IL-10, CD4, FOXP3, and any combination thereof.

In some aspects, the neurological biomarkers are selected from CGRP, ChAT, VAChT, and any combination thereof.

In some aspects, the methods comprise detecting the presence and/or absence and/or measuring the level of expression of at least one neurological biomarker in the subject and determining whether or whether not to treat the subject with a checkpoint inhibitor therapy disclosed herein (e.g., an anti-PD-1 antibody). In some aspects, the presence and/or absence and/or expression level of the neurological biomarker or a plurality of neurological biomarkers is determined according methods disclosed herein. In some aspects, the presence and/or absence and/or expression level of a neurological biomarker in the subject sample is an indication that the subject is a responsive subject and would benefit from a checkpoint inhibitor therapy disclosed herein (e.g., an anti-PD-1 antibody). In some aspects, the presence and/or absence and/or expression level of a plurality of neurological biomarkers in the subject is an indication that the subject is a responsive patient and would benefit from a checkpoint inhibitor therapy disclosed herein (e.g., an anti-PD-1 antibody). In some aspects, a predictive biomarker for use in the methods comprises a biomarkers which demonstrate a presence and/or increased expression in a subject suffering from a cancer or tumor (e.g., cSCC).

In some aspects, the disclosure further provides methods for determining whether to treat or not treat a subject suffering from a cancer or a tumor (e.g., cSCC). Such methods comprise measuring the presence and/or absence and/or level of expression of at least one neurological biomarker in the subject and determining whether or whether not to treat the subject with a combination therapy as described herein based on the presence and/or absence and/or expression level of the neurological biomarker or plurality of neurological biomarkers. In some aspects, the presence and/or absence and/or expression level of a neurological biomarker in the subject sample is an indication that the subject would or would not benefit, respectively, from the combination therapy and/or immune checkpoint inhibitor (e.g., anti-PD-1) therapy as described herein.

### IV. Combination Therapy

Certain aspects of the disclosure are directed to a combination therapy comprising a molecule that targets an immune checkpoint protein (e.g., programmed death 1 (PD-1)) (e.g., an antibody that specifically binds to immune checkpoint protein (e.g., PD-1)) and a molecule that targets a neurological biomarker (e.g., antibody that specifically binds to a neurological biomarker).

Certain aspects of the disclosure relate to the combination therapy comprising: an antibody that specifically binds to immune checkpoint protein (e.g., programmed death 1 (PD-1)); and an antibody that specifically binds to a neurological biomarker selected from the group consisting of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription Factor 3 (ATF3), cJUN, Calcitonin Gene-Related Peptide (CGRP), Choline Acetyltransferase (ChAT), and Vesicular Acetylcholine Transporter (VAChT).

Programmed Death-1 (PD-1) receptor refers to an immunoinhibitory receptor belonging to the CD28 family. PD-1 is expressed predominantly on activated T cells in vivo, and binds to two ligands, PD-L1 and PD-L2. PD-1 includes human PD-1 (hPD-1), variants, isoforms, and species homologs of hPD-1, and analogs having at least one common epitope with hPD-1. The hPD-1 sequence can be found under GENBANK® Accession No. U64863.

Engagement of PD-1 with its known binding ligands, PD-L1 and PD-L2, occurs primarily within the tumor microenvironment and results in downregulation of anti-tumor specific T-cell responses. Both PD-L1 and PD-L2 are known to be expressed on tumor cells. The expression of PD-L1 and PD-L2 on tumors has been correlated with decreased survival outcomes.

CTLA4 is a member of the immunoglobulin superfamily, which is expressed on the surface of Helper T cells and transmits an inhibitory signal to T cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal.

Like the ligands of CD28 and CTLA-4, PD1 ligand (PD-L1 and PD-L2) are B7 family proteins comprised of tandem V-set and C1-set IgSF domains. In addition to PD-1, PD-L1 binds B7-1, one of the ligands of CD28 and CTLA-4, potentially interlocking the PD-1 and CD28/CTLA-4 signaling pathways. Regulatory T lymphocytes express a large amount of CD28 and of CTLA4 that prevent or allow, respectively, the suppressive activity of regulatory T lymphocytes. In the presence of antigen presenting cells (APC) expressing high level of B7, the CD28/B7 interaction prevents the suppressive activity of regulatory T lymphocytes (Sansom et al., Trends Immunol. 24, 314-319, 2003).

LAG3 is expressed by T cells, B cells, NK cells and plasmacytoid dendritic cells (pDCs) and is upregulated following T cell activation. It modulates T cell function as well as T cell homeostasis. Subsets of conventional T cells that are anergic or display impaired functions express LAG3. LAG3+ T cells are enriched at tumor sites and during chronic viral infections (Sierro et al Expert Opin. Ther. Targets 15 (2011), 91-101). It has been shown that LAG3 plays a role in CD8 T cell exhaustion (Blackburn et al. Nature Immunol. 10 (2009), 29-37). Thus, there is a need for antibodies that antagonize the activity of LAG3 and can be used to generate and restore immune response to tumors.

4-1BB (CD137 and TNFRSF9), was first identified as an inducible costimulatory receptor expressed on activated T cells, is a membrane spanning glycoprotein of the Tumor Necrosis Factor (TNF) receptor superfamily. 4-1BB indicates that expression is generally activation dependent and encompasses a broad subset of immune cells including activated NK and NKT cells, regulatory T cells, dendritic cells (DC) including follicular DC; stimulated mast cells, differentiating myeloid cells, monocytes, neutrophils, eosinophils, and activated B cells. 4-1BB expression has also been demonstrated on tumor vasculature and atherosclerotic endothelium. The ligand that stimulates 4-1BB (4-1BBL) is expressed on activated antigen-presenting cells (APCs), myeloid progenitor cells and hematopoeitic stem cells.

OX40 (also known as CD34, TNFRSF4 and ACT35) is a member of the tumor necrosis factor receptor superfamily. OX40 is not constitutively expressed on naive T cells, but is induced after T Cell Receptor (TCR) involvement. The ligand OX40L of OX40 is expressed predominantly on antigen presenting cells. OX40 is highly expressed by activated CD4+ T cells, activated CD8+ T cells, memory T cells, and regulatory T cells. OX40 signaling can provide co-stimulatory signals to CD4 and CD8T cells, resulting in enhanced cell proliferation, survival, effector function and migration. OX40 signaling also enhances memory T cell development and function.

CD27 typically exists as a glycosylated, type I transmembrane protein, frequently in the form of homodimers with a disulfide bridge linking the two monomers. Crosslinking the CD27 antigen on T cells provides a costimulatory signal that, in concert with T-cell receptor crosslinking, can induce T-cell proliferation and cellular immune activation. CD27 is expressed on mature thymocytes, on most CD4+ and CD8+ peripheral blood T cells, natural killer cells and B cells (Kobata T, et al., Proc. Natl. Acad. Sci. USA. 1995 Nov. 21; 92(24):11249-53). CD27 is also highly expressed on B cell non-Hodgkin's lymphomas and B cell chronic lymphocytic leukemias (Ranheim E A, et al., Blood. 1995 Jun. 15; 85(12):3556-65). Additionally, increased levels of soluble CD27 protein have been identified in sera or sites of disease activity in parasitic infection, cytomegalovirus (CMV) infection, sarcoidosis, multiple sclerosis, and B-cell chronic lymphocytic leukemia (Loenen W A, et al., Eur. J. Immunol. 22:447, 1992).

T cell immunoglobulin and mucin domain-containing molecule 3 (TIM3) is an immunoglobulin (Ig) superfamily member, expressed on Th1 cells. TIM3 has been shown to play a role in modulating the immune response of Th1 cells, and reducing inflammation in a number of conditions. TIM3 is also expressed on cancer cells, and on cancer stem cells (CSCs), which are cells that can give rise to additional cancer cells.

In some aspects, an antibody that specifically binds to a protein in an immune checkpoint pathway. In some aspects, the antibody disclosed herein binds an immune checkpoint protein (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), LAG3 antagonist (e.g., anti-LAG3 antibody), 4-1BB antagonist (anti-4-1BB antibody), OX-40 antagonist (anti-OX-40 antibody), CD27 antagonist (e.g., anti-CD27 antibody), CD28 antagonist (e.g., anti-CD28 antibody), B7-H3 antagonist (e.g., anti-B7-H3 antibody), TIM-3 antagonist (e.g., anti-TIM3 antibody), or combinations thereof.

A review describing immune checkpoint pathways and the blockade of such pathways with immune checkpoint inhibitor compounds is provided by Pardoll in Nature Reviews Cancer (April, 2012), pages 252-264. Immune checkpoint inhibitor compounds display anti-tumor activity by blocking one or more of the endogenous immune checkpoint pathways that downregulate an anti-tumor immune response. The inhibition or blockade of an immune checkpoint pathway typically involves inhibiting a checkpoint receptor and ligand interaction with an immune checkpoint inhibitor (e.g. PD-1) to reduce or eliminate the down regulation signal and resulting diminishment of the anti-tumor response.

In some aspects of the present disclosure, the immune checkpoint inhibitor compound inhibits the signaling interaction between an immune checkpoint receptor and the corresponding ligand of the immune checkpoint receptor. The immune checkpoint inhibitor compound can act by blocking activation of the immune checkpoint pathway by inhibition (antagonism) of an immune checkpoint receptor (some examples of receptors include PD-1,) or by inhibition of a ligand of an immune checkpoint receptor (some examples of ligands include PD-L1 and PD-L2). In such aspects, the effect of the immune checkpoint inhibitor compound is to reduce or eliminate down regulation of certain aspects of the immune system anti-tumor response in the tumor microenvironment.

In some aspects, the disclosed methods include administering a therapeutically effective amount of an immune checkpoint inhibitor (e.g., an anti-PD-1 antibody) in combination with an antibody directed to a neurological biomarker. In some aspects, the PD-1 inhibitor is administered before, after, or concurrent with the antibody directed to a neurological biomarker.

Concurrent administration of the immune checkpoint inhibitor (e.g., an anti-PD-1 antibody) and antibody directed towards a neurological biomarker does not require that the agents be administered at the same time or by the same route, as long as there is an overlap in the time period during which the agents are exerting their therapeutic effect. Simultaneous or sequential administration is contemplated, as is administration on different days or weeks.

In some aspects, the immune checkpoint inhibitor (e.g., an anti-PD-1 antibody) and the antibody directed to a neurological biomarker are in the same composition.

In some aspects, the immune checkpoint inhibitor (e.g., an anti-PD-1 antibody) and the antibody directed to a neurological biomarker are in separate compositions.

In some aspects, the immune checkpoint inhibitor (e.g., an anti-PD-1 antibody) is suitable for intratumorally, intravenously, intraperitoneally, intramuscularly, intrathecally or subcutaneous delivery.

In some aspects, the immune checkpoint inhibitor (e.g., an anti-PD-1 antibody) is suitable for intravenous delivery.

In some aspects, the antibody directed to a neurological biomarker is suitable for intratumorally, intravenously, intraperitoneally, intramuscularly, intrathecally or subcutaneous delivery.

In some aspects, the combination therapy disclosed herein comprising the immune checkpoint inhibitor (e.g., an anti-PD-1 antibody) and the antibody directed to a neurological biomarker is suitable for intratumorally, intravenously, intraperitoneally, intramuscularly, intrathecally or subcutaneously delivery in the same composition.

The combination therapy disclosed herein can be administered by any route which results in a therapeutically effective outcome. In some aspects, the methods disclosed herein can comprise re-administering a combination therapy of the present disclosure to the subject.

In some aspects, the antibody that specifically binds to an immune checkpoint protein is selected from the group consisting of an anti-PD-1 antibody, anti-CTLA-4 antibody, anti-LAG-3 antibody, anti-4-1BB antibody, anti-OX-40 antibody, anti-CD27 antibody, anti-CD28 antibody, anti-TIM3 antibody, anti-B7-H3 antibody, VISTA antibody, anti-IDO-1 antibody, and any combination thereof.

In some aspects, the combination therapy comprises a molecule that targets PD-1. As used herein, an example of therapies that target PD-1 can include: Pembrolizumab (Keytruda), Nivolumab (Opdivo), Dostarlimab (Jemperli) or Cemiplimab (Libtayo). These drugs have been shown to be helpful in treating several types of cancer, and new cancer types are being added as more studies show these drugs to be effective.

As used herein, an immune checkpoint inhibitor that specifically binds to programmed death 1 (PD-1) is selected from a group consisting of nivolumab, pembrolizumab, cemiplimab, and dostarlimab.

In some aspects, the combination therapy comprises a molecule that targets a neurological biomarker, e.g., an antibody that binds one or more of NfH, NfL, TH, GFAP, OLIG2, CD39, ATF3, and cJUN.

In some aspects, the combination therapy comprises a molecule that targets a neurological biomarker, e.g., an antibody that binds one or more of NfH, NfL, TH, GFAP, OLIG2, CD39, ATF3, cJUN, CGRP, ChAT, and VAChT.

In some aspects, the methods of the present disclosure comprise administering a therapeutically effective amount of an immune checkpoint inhibitor (e.g., an anti-PD-1 antibody) and a neurological biomarker inhibitor (e.g., an antibody that binds NfH, NfL, TH, GFAP, OLIG2, CD39, ATF3, or cJUN) to a subject suffering from cancer.

In some aspects, the methods of the present disclosure comprise administering a therapeutically effective amount of an immune checkpoint inhibitor (e.g., an anti-PD-1 antibody) and a neurological biomarker inhibitor (e.g., an antibody that binds NfH, NfL, TH, GFAP, OLIG2, CD39, ATF3, cJUN, CGRP, ChAT, or VAChT) to a subject suffering from cancer.

In some aspects other cancers that can be treated using the methods of the present disclosure include breast cancer, bladder cancer, cervical cancer, colon cancer, head and neck cancer, Hodgkin lymphoma, liver cancer, lung cancer, renal cell cancer (a type of kidney cancer), skin cancer (including melanoma, squamous cell carcinoma, or a cutaneous squamous cell carcinoma (cSCC)), stomach cancer, rectal cancer.

The methods and materials provided herein can be used to treat any appropriate type of cancer. For example, the methods and materials provided herein can be used to treat breast cancer, bladder cancer, cervical cancer, colon cancer, head and neck cancer, Hodgkin lymphoma, liver cancer, lung cancer, renal cell cancer (a type of kidney cancer), skin cancer (including melanoma, squamous cell carcinoma, or a cutaneous squamous cell carcinoma (cSCC)), stomach cancer, rectal cancer

In some aspects, the cSCC is a solid tumor (e.g., an advanced solid tumor). In some aspects, the cSCC is a reoccurring tumor. In some aspects, the cSCC is a metastatic tumor. In some aspects, the advanced cSCC solid tumor is indolent or aggressive. In some aspects, the subject is not responsive to, or has relapsed (e.g., experienced a recurrent lesion) after, prior therapy or surgery.

In some aspects, administration of the combination therapy disclosed herein treats or inhibits the recurrence of tumors in a subject with cancer.

In some aspects, administration of the combination therapy disclosed herein treats or inhibits the recurrence of tumors in a subject with cSCC.

### V. Method of Treatment

Certain aspects of the disclosure provide a method of treating a subject suffering from cancer (e.g., cutaneous squamous cell carcinoma (cSCC)) comprising administering to the subject a therapeutically effective amount of a combination therapy comprising (i) an antibody that specifically binds to an immune checkpoint protein (e.g., a programmed death 1 (PD-1)), and (ii) an antibody directed to a neurological biomarker disclosed herein.

In some aspects, the cancer is selected from the group consisting of breast cancer, bladder cancer, cervical cancer, colon cancer, head and neck cancer, Hodgkin lymphoma, liver cancer, lung cancer, renal cell cancer (a type of kidney cancer), skin cancer (including melanoma, squamous cell carcinoma, or a cutaneous squamous cell carcinoma (cSCC)), stomach cancer, rectal cancer, or any combination thereof.

In some aspects, the cSCC is selected from the group consisting of advanced cSCC, metastatic cSCC, locally advanced cSCC, resectable cSCC, unresectable cSCC, recurrent cSCC, and any combination thereof.

The cSCC can be a recurrent cancer. In some aspects, the cancer is recurrent if the subject has a repeated diagnosis of the cSCC or a frequent or repeated occurrence of a related skin lesion(s) or metastasis, such as regrowth of a primary tumor lesions and/or new tumor lesions that may represent recurrence of the prior tumor lesion.

In some aspects, if the subject is identified as exhibiting an absence and/or a low level of a neurological biomarker, the subject is not administered a therapy comprising (i) an antibody that specifically binds to an immune checkpoint protein (e.g., programmed death 1 (PD-1)), and/or (ii) an antibody directed to a neurological biomarker disclosed herein.

In some aspects, if the subject is identified as exhibiting a presence and/or a high level of a neurological biomarker, the subject is administered a therapy comprising (i) an antibody that specifically binds to an immune checkpoint protein (e.g., programmed death 1 (PD-1)), and/or (ii) an antibody directed to a neurological biomarker disclosed herein.

In some aspects, the administration of the antibody directed to an immune checkpoint protein (e.g., anti-PD-1 antibody) and/or the antibody directed to a neurological biomarker inhibits the recurrence of a lesion (e.g., a cSCC tumor).

In some aspects, the immune checkpoint inhibitor (e.g., anti-PD-1 antibody) and/or the antibody directed to a neurological biomarker is suitable for intratumorally, intravenously, intraperitoneally, intramuscularly, intrathecally or subcutaneous delivery.

In some aspects, the immune checkpoint inhibitor (e.g., anti-PD-1 antibody) and/or the antibody directed to a neurological biomarker is suitable for intravenous delivery.

In some aspects, the combination therapy disclosed herein comprising the immune checkpoint inhibitor (e.g., anti-PD-1 antibody) and the antibody directed to a neurological biomarker is suitable for intratumorally, intravenously, intraperitoneally, intramuscularly, intrathecally or subcutaneously delivery in the same composition.

Certain aspects of the disclosure are directed to a method of treating a subject suffering from cancer comprising administering to the subject a therapeutically effective amount of a combination therapy disclosed herein to the subject.

Certain aspects of the disclosure are directed to a method of treating a subject suffering from cutaneous squamous cell carcinoma (cSCC) comprising administering to the subject a therapeutically effective amount of a combination therapy disclosed herein to the subject.

In some aspects, the immune checkpoint inhibitor (e.g., anti-PD-1 antibody) and the antibody directed to a neurological biomarker are administered simultaneously or sequentially.

In some aspects, the immune checkpoint inhibitor (e.g., anti-PD-1 antibody) and the antibody directed to a neurological biomarker are administered in the same composition.

In some aspects, the immune checkpoint inhibitor (e.g., anti-PD-1 antibody) and the antibody directed to a neurological biomarker are administered in different compositions.

In some aspects, the immune checkpoint inhibitor (e.g., anti-PD-1 antibody) is administered prior to or at the time of administration of the antibody directed to a neurological biomarker.

The present disclosure also provides kits, or products of manufacture, comprising (i) the combination therapy as disclosed herein, and (ii) optionally instructions for use (e.g., a package insert with instructions to perform any of the methods described herein).

In some aspects, the kit or product of manufacture comprises (i) an antibody that specifically binds to an immune checkpoint (e.g., anti-PD-1 antibody) (ii) an antibody that specifically binds to a neurological biomarker selected from the group consisting of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription Factor 3 (ATF3), and cJUN; and (iii) optionally, instructions for use (e.g., a package insert with instructions to perform any of the methods described herein are also contemplated).

In some aspects, the kit or product of manufacture comprises (i) an antibody that specifically binds to an immune checkpoint (e.g., anti-PD-1 antibody) (ii) an antibody that specifically binds to a neurological biomarker selected from the group consisting of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1 (CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription Factor 3 (ATF3), cJUN, and Calcitonin Gene-Related Peptide (CGRP), Choline Acetyltransferase (ChAT), and Vesicular Acetylcholine Transporter (VAChT); and (iii) optionally, instructions for use (e.g., a package insert with instructions to perform any of the methods described herein are also contemplated).

One skilled in the art will readily recognize that vectors, polynucleotides, and pharmaceutical compositions of the present disclosure, or combinations thereof, can be readily incorporated into one of the established kit formats which are well known in the art.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

The following examples are illustrative and do not limit the scope of the claimed aspects.

### EXAMPLES

### Example 1. Pre and Post Treatment Response

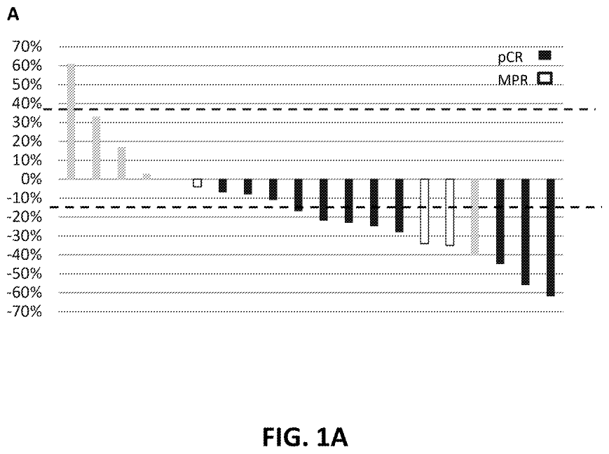

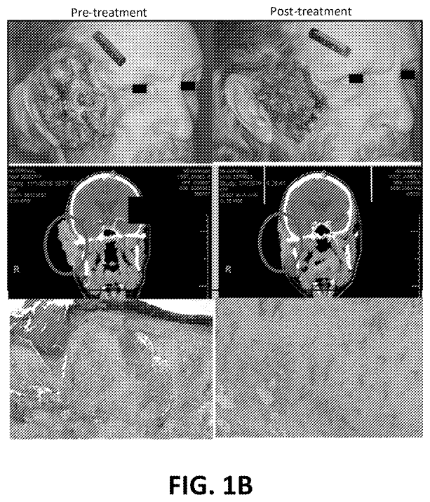

FIG. 1A shows the waterfall plot of percentage change from baseline in target lesions per RECIST 1.1 and coded for the pathological complete response (PCR) and the major pathological response (MPR) following the neoadjuvant cemiplimab treatment.

FIG. 1B shows the representative pretreatment (left) and post-treatment (right) photographs, coronal computed tomography (CT) images, and micrographs of tumor specimens of a subject who achieved a major pathologic response (6-mm foci of residual viable tumor) following neoadjuvant cemiplimab treatment.

### Example 2. Staining of Biomarkers

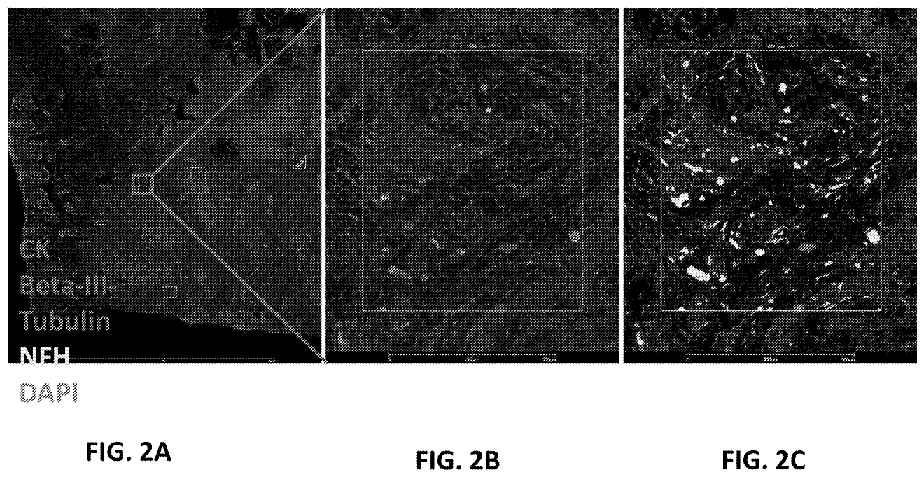

FIGS. 2A-2C show DAPI staining of Creatine Kinase (CK), Beta-III-Tubulin, Neurofilament Heavy chain (NfH) at 500 μm. The DAPI staining can be completed using a immunofluorescence technique on the Nanostring digital spatial profiling (DSP) platform. DAPI staining can be performed by rinsing the samples above twice in phosphate buffered saline (PBS) at concentrations ranging between 1 -0.1 μg/ml, for five minutes each. Then incubating samples can be kept at room temperature in the absence of light source. Samples can then be rinsed in PBS and prepared for imaging. Examination can then be performed using the appropriate excitation wavelength (500 μm) and results in FIGS. 2A-2C.

In this example, the DAPI staining was performed using formalin-fixed and paraffin-embedded tissue from surgical resected head and neck squamous cell carcinoma samples. Two serial 5 μm thick sections were then stained with the semi-automated GeoMx Digital Spatial Profiling (DSP) standard protein protocol. The first stain using the Leica BOND RX autostainer system (Leica Biosystem) to profile tumor associated nerves and its perineural microenvironment. Both sections were then stained with the following morphology biomarkers: pancytokeratin (panCK) and SYTO 13 (GeoMx Solid Tumor TME Morphology Kit, cat#121300301), and B3-tubulin (clone EP1569Y, AF, catalog number: ab52623, Abcam, concentration 1:2000 μg/ml) and neurofilament (clone EPR20020, AF, Abcam, catalog number: ab207176, concentration 1:1000 μg/ml).

Optimization of immunofluorescence biomarkers was performed with different dilutions using normal colon tissue to achieve the highest signal to noise ratio. One tumor section was used to profile tumor associated nerves using the following DSP neuro protein cores and modules: neural cell profiling core (cat#GMX-PROCO-NCT-HNCP-12) and GeoMx Parkinson's pathology panel (cat#GMX-PROMOD-NCT-HPDP-12).

A serial section was used to profile the perineural microenvironment with the following DSP human immuno-oncology protein core and modules were used: GeoMx Immune Cell Profiling (cat#121300101), GeoMx IO Drug Target Assay (cat#121300102), GeoMx Immune Activation Status Assay (cat#121300103), Immune Cell Typing Assay (cat#121300104) (49 protein targets).

Selections of region of interest (ROI) were then performed after pathology evaluation of sequential sections of hematoxylin & eosin staining, nerve fibers in tumor tissue and tumor bed tissue were then identified by an expert pathologist (PN).

### Example 3. Responders Pre-Treatment and Non-Responders Pre-Treatment Dendrogram

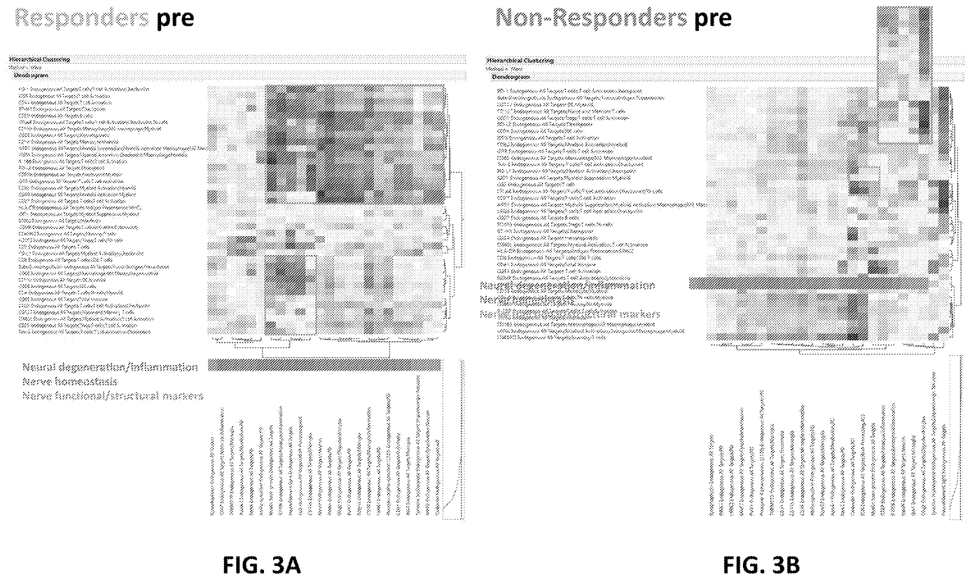

FIGS. 3A-3B shows a dendrogram of potential biomarkers detailing responders and non-responders pre-treatment of a neoadjuvant cemiplimab treatment. The results in FIGS. 3A-3B were generated by creating a Pearson's correlation matrix from the digital spatial profiling DSP protein expression data, between immune and neurological biomarkers within the pre-treatment responders and pre-treatment non-responders.

### Example 4. Responders Post-Treatment and Non-Responders Post-Treatment Dendrogram

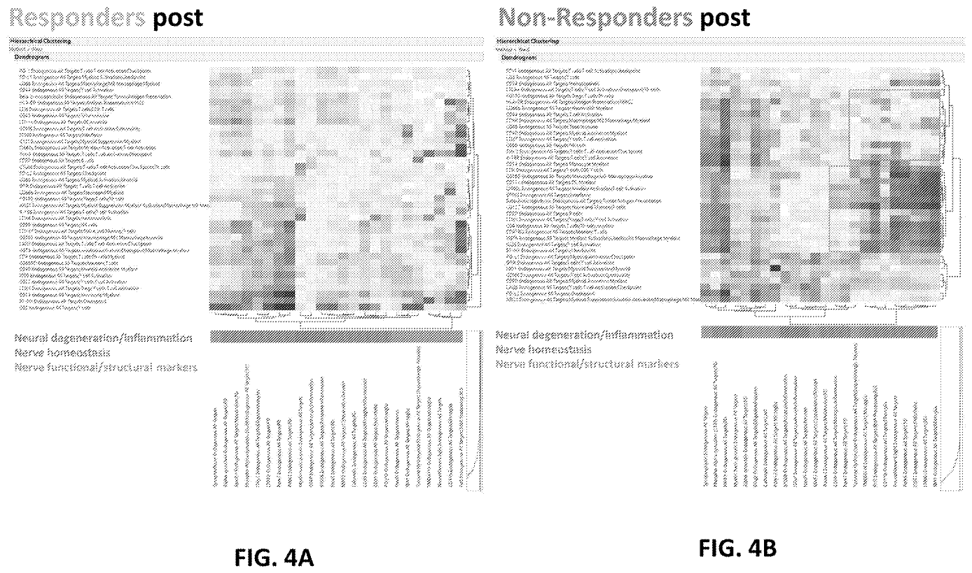

FIGS. 4A-4B shows a dendrogram of potential biomarkers detailing responders and non-responders post-treatment of a neoadjuvant cemiplimab treatment. The results in FIGS. 4A-4B were generated by creating a Pearson's correlation matrix from the digital spatial profiling DSP protein expression data, between immune and neurological biomarkers within the post-treatment responders and post-treatment non-responders.

### Example 5. Microbiome Chart

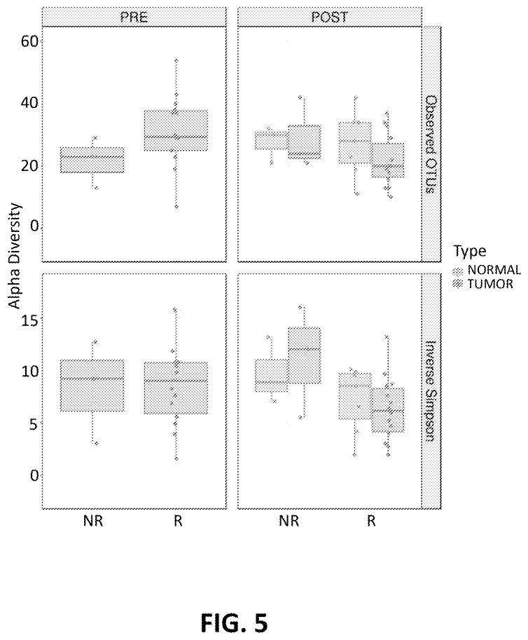

FIG. 5 shows the alpha diversity of the microbiome in a subject's tumor pre and post treatment generated using the 16S rRNA gene sequencing technique, which was then used to identify, classify and quantify microbes within the complex biological mixtures such as samples collected from the subject's tumor, pre and post treatment. The region of choice (e.g. V4, V1-V3) is PCR amplified and molecular barcodes are generated, which allows multiple samples to be sequenced together. Barcodes may be incorporated as part of PCR amplification or as a separate step during library preparation. Libraries are then sequenced to the desired depth using either a single-end or paired-end protocol. Resulting reads are separated on the basis of the barcode index (demultiplexing). Due to common errors that are introduced during PCR amplification and sequencing, certain quality control steps are required in order to reduce false positives and misinterpretation of results. These include quality filtering, chimera detection and removal, and strict merging conditions (for paired-end protocols).

Once the quality control steps were completed, the large number of sequences were partitioned into meaningful units prior to taxonomic assignment. This was achieved using a clustering approach. Operational taxonomic unit (OTU) clustering (e.g. via the UPARSE algorithm) primarily relied on computing distance matrices from pairwise alignments in order to group sequences at a user specified threshold (typically ≥97%) and identifying a representative sequence which was used for taxonomic identification.

Representative sequences were then mapped against well-curated 16S rRNA gene databases such as SILVA to determine taxonomy assignment. Together with the number of sequence reads present per OTU, this information provided an overview of community composition as well as an estimate of the relative abundance of individual taxa within that community.

### Example 6 Cancer-Induced Nerve Damage was Associated With Poor Clinical Response to Anti-PD-1 Therapy

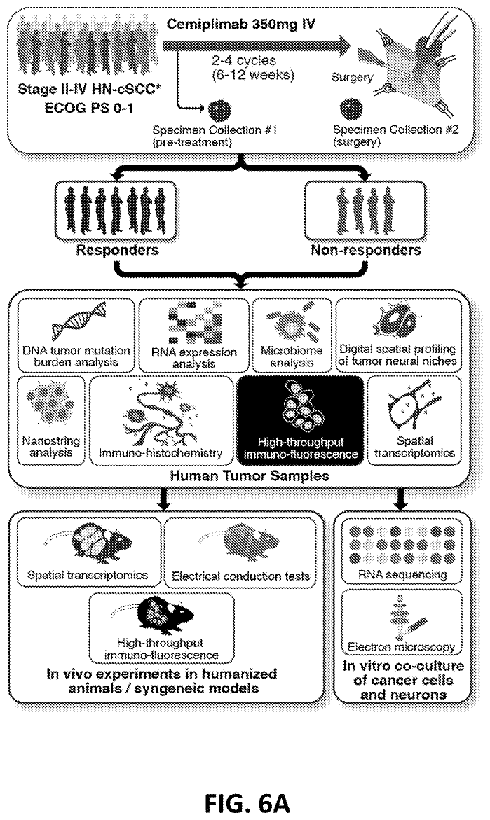

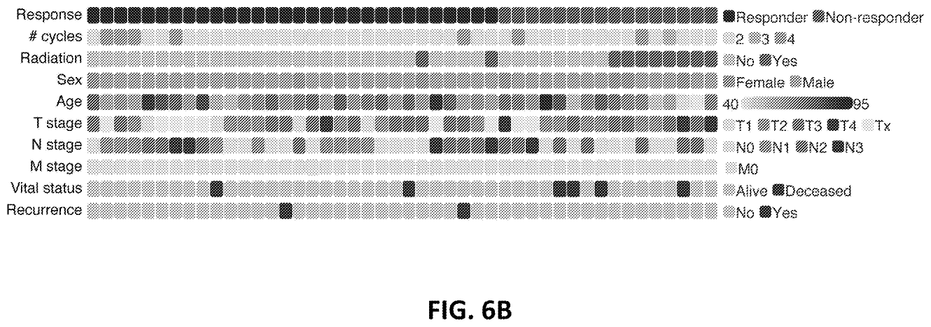

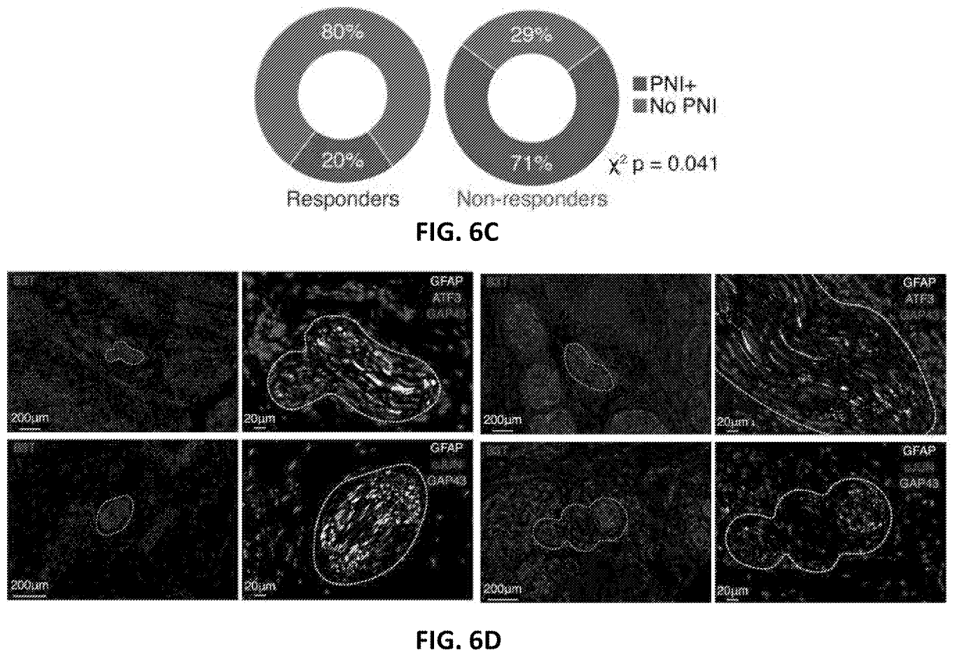

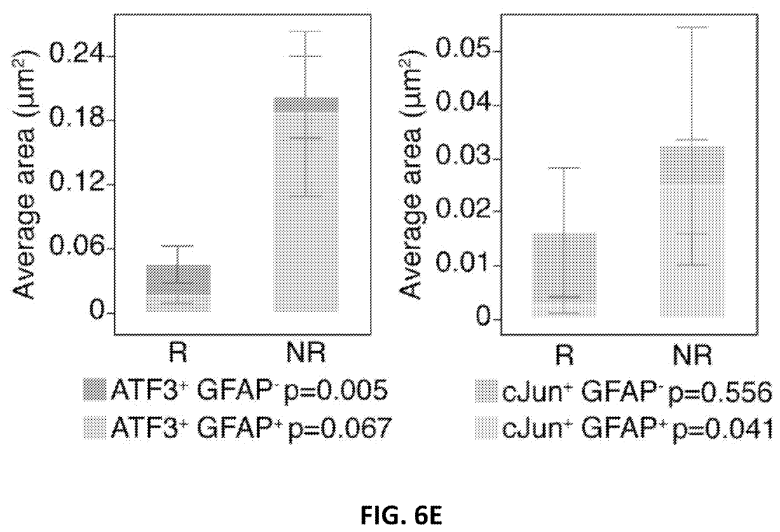

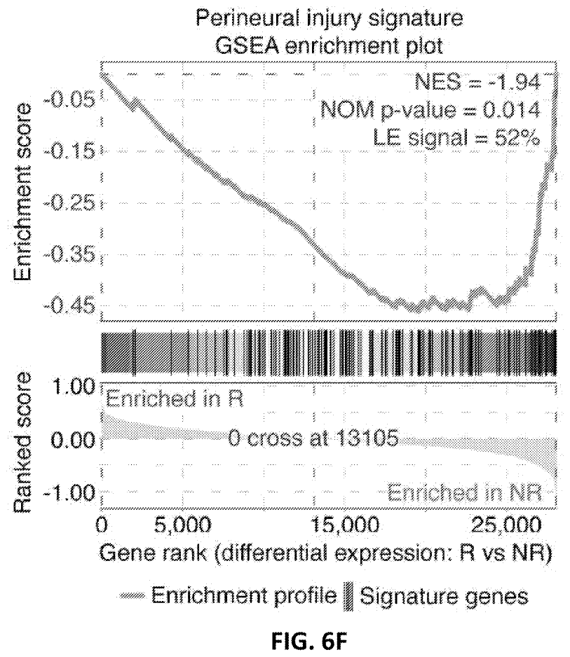

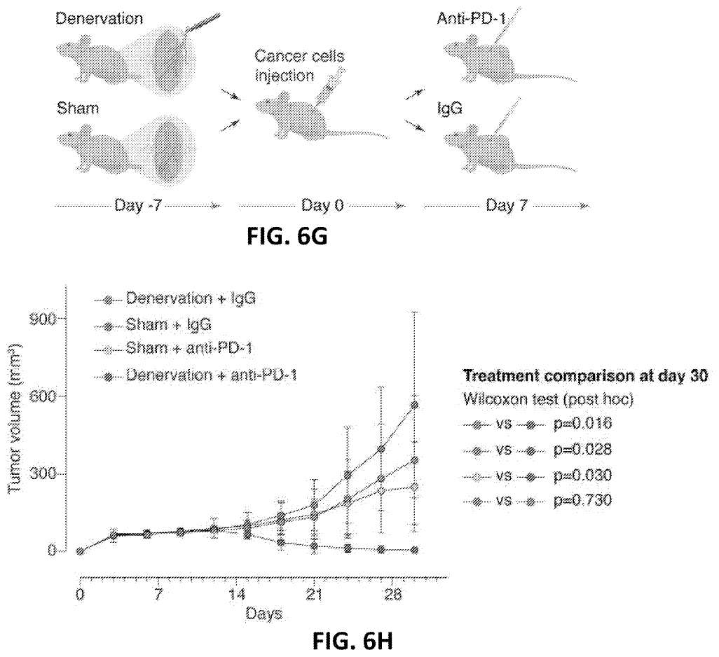

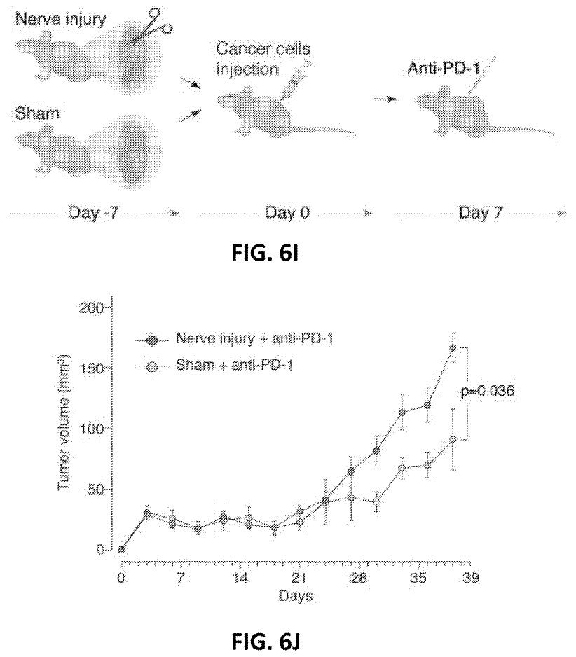

To evaluate the role of tumor-associated nerves (TANs) in clinical response to anti-PD- 1 therapy, tumor samples from 55 patients with stage II-IVA cutaneous squamous cell carcinoma (cSCC) were utilized. Samples were from clinical trials NCT03565783 and NCT04154943. All patients from these clinical trials underwent neoadjuvant anti-PD-1 therapy administration (Cemiplimab) followed by surgery (FIG. 6A).

For this study, radiation treatment was not administered prior to the anti-PD-1 therapy. At least two cycles of Cemiplimab were administered throughout the trial to all patients. Within one trial (NCT04154943), the patients were allowed to receive up to 4 cycles of neoadjuvant Cemiplimab if they did not progress radiologically or clinically, and tolerated the treatment (FIG. 6B). The responders (n=31) were defined as patients with less than 10% viable tumor cells at surgery. The non-responders (n=16) were defined as patients with more than 50% viable tumor cell in the neoadjuvant-treated surgical specimens.

Patients who exhibited 10%-50% viable tumor cells in the surgical specimens (n=8) were excluded, as the patient population was noted to previously exhibit inconsistent assignment to both the responders and non-responders groups in neoadjuvant clinical trials.

The initial step in examining the potential role of TANs in clinical response to anti-PD-1 therapy was to assess tumors for the presence of peripheral nerve invasion (PNI). PNI is a histo-morphological phenomenon, defined as the presence of tumor cells abutting or in close proximity to a nerve with encirclement of at least a third of the nerve circumference by tumor; or the presence of cancer cells within the epineurial, perineurial, and/or endoneurial compartments of a nerve.

At baseline, non-responders had a significantly higher incidence of PNI compared to responders (71% versus 20%, respectively, p=0.041, see FIG. 6C). To test whether cancer cells damaged the invaded nerves, expression of canonical nerve response to injury markers ATF3 and c-Jun in the neural niches was quantified (FIG. 6D). Analysis of baseline tumor samples (responders n=7, non-responders n=6, see FIG. 6E) revealed higher expression levels of both Schwan (i.e., GFAP+) c-Jun, and neuronal (i.e., GFAP−) ATF3 (p=0.041 and p=0.005, respectively) in non-responders compared to responders.

Transcriptional alterations were over-expressed in neoadjuvant treated tumors of non-responders compared to responders (FDR 0.014, see FIG. 6F).

To evaluate whether TAN damage may promote resistance to anti-PD-1 therapy, two neuromodulated cSCC mouse models were studied. First, the nerves from the tumor micro environment “TME” were excised by plucking the nerves innervating the skin of immunocompetent SKH1-Elite (SKH1-Hrhr, Charles River) mice. Denervation was then confirmed by histology. Sham surgery was performed in the control group (FIG. 6G). Skin denervation was confirmed one week post-denervation using behavioral testing. SCC cells (B6, ultraviolet induced, SKH1-Hrhr derived) were orthotopically injected to the denervated skin. Seven days after cancer inoculation, mice were treated with either anti-PD-1 or IgG2 control. Denervated mice demonstrated improved tumor response to anti-PD-1 therapy with significantly lower tumor volumes compared to the control groups (P=0.03, see FIG. 6H).

Next, the impact of TAN damage in response to anti-PD-1 was evaluated. Nerve damage was induced using surgical axotomy (FIG. 6I). Within the mouse model, severed nerves were left resting in place, which subsequently resulted in Wallerian degeneration (anterograde disintegration of axons and their transected myelin sheaths). Then, one-week post-axotomy, cutaneous B6 SCC cells were orthotopically injected into a numb dermatome, followed by treatment with anti-PD-1. Axotomized mice exhibited a significantly detrimental tumor control compared to sham control mice (P=0.036, see FIG. 6J).

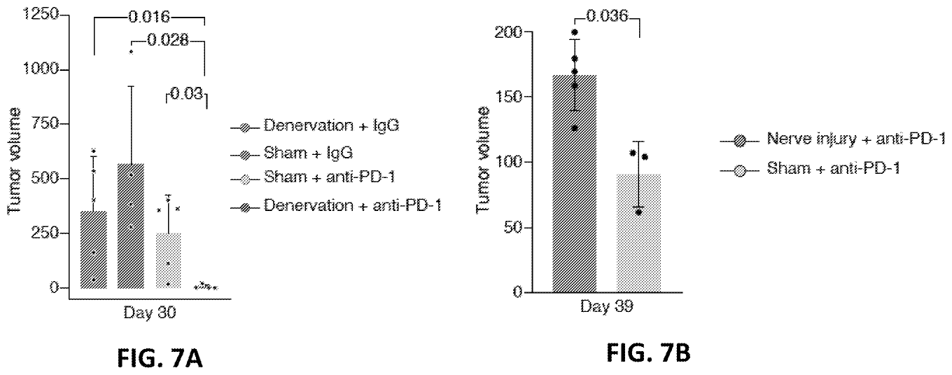

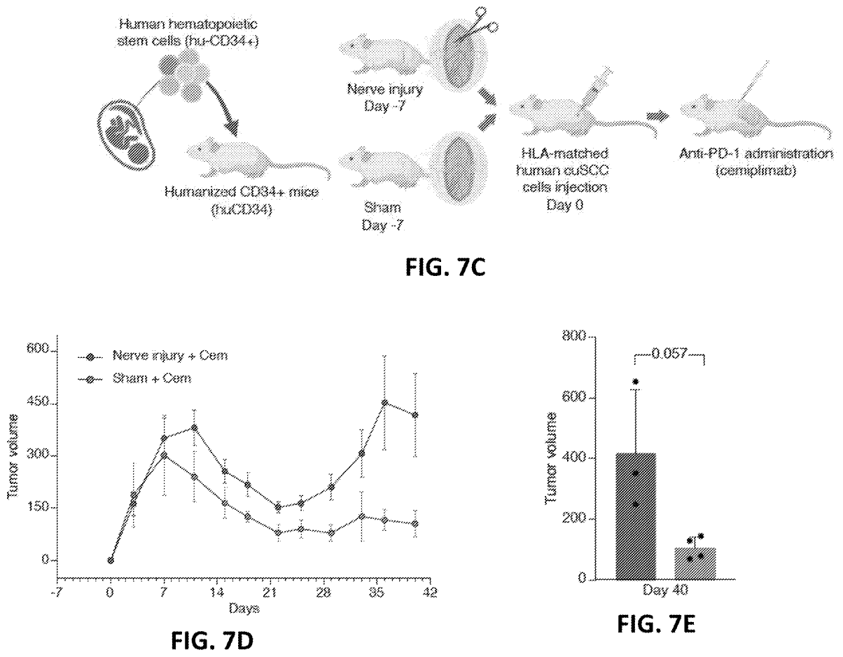

To assess potential drug-specific effects of Cemiplimab on human immune cells, this experiment was repeated with human leukocyte antigen-matched human cutaneous SCC cells (IC845) that were injected into axotomized or sham operated skin of humanized CD34+ NOD-scid gamma (huCD34-NSG) mice. Initially both groups demonstrated a response to Cemiplimab, however, only sham operated mice had a durable response (417.7 mm3 versus 105.4 mm3 in axotomized and sham operated mice, respectively, P=0.057 on day 40), (FIG. 7A-FIG. 7E).

### Example 7 Cancer Cells Damage Nerves by Inducing Nerve Demyelination and Degeneration

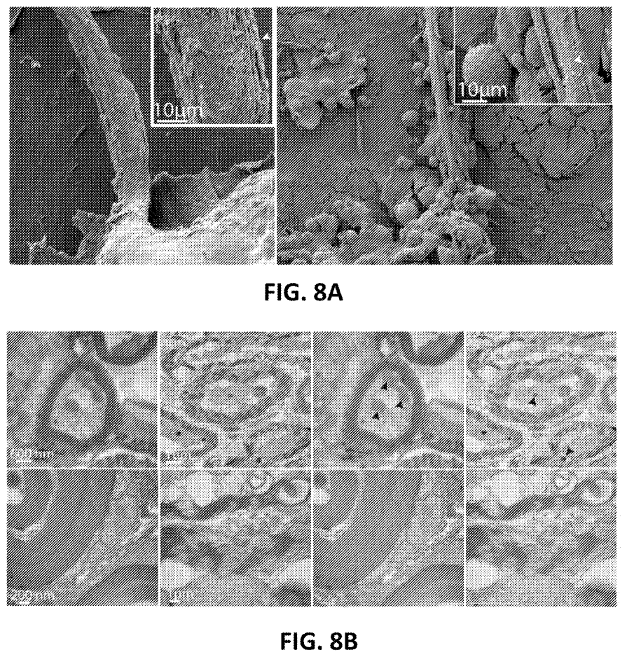

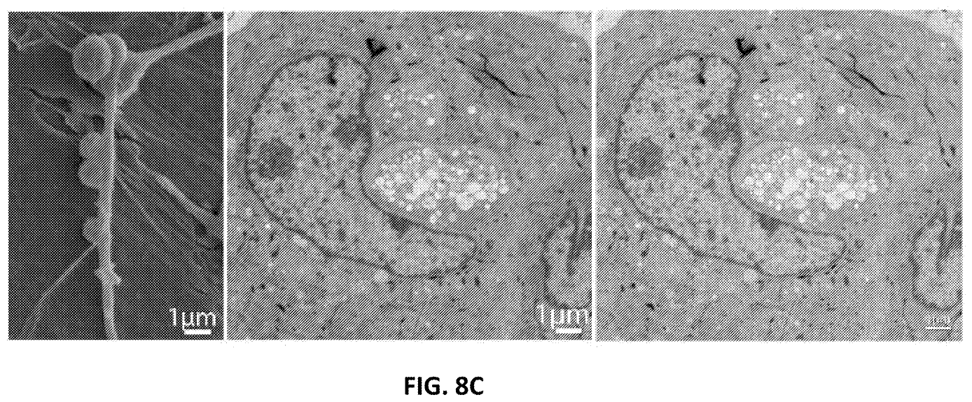

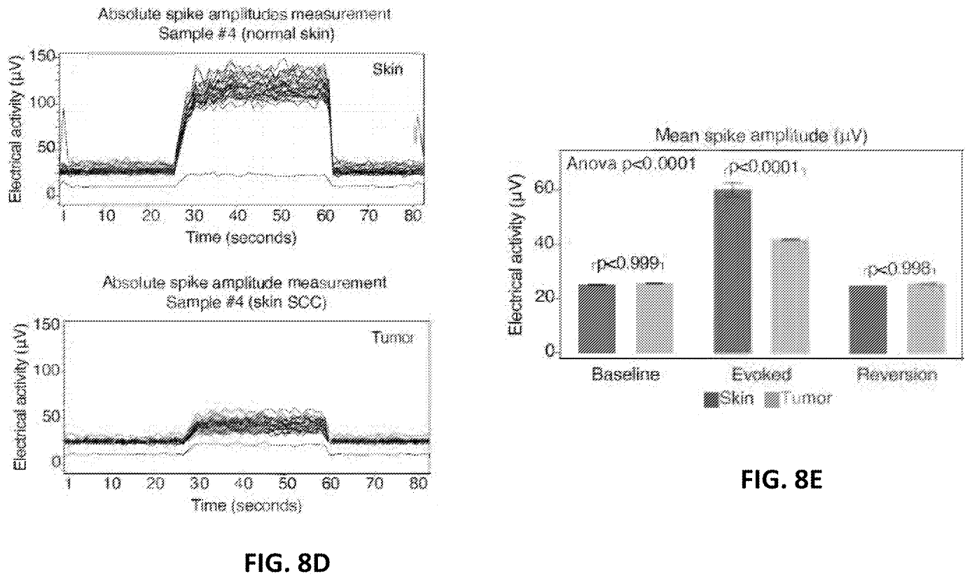

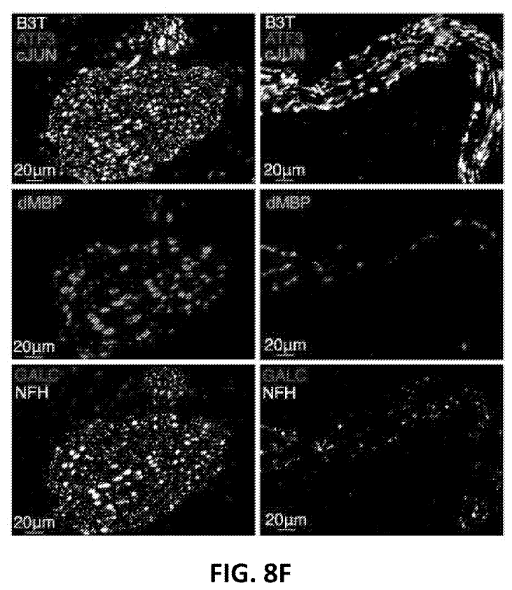

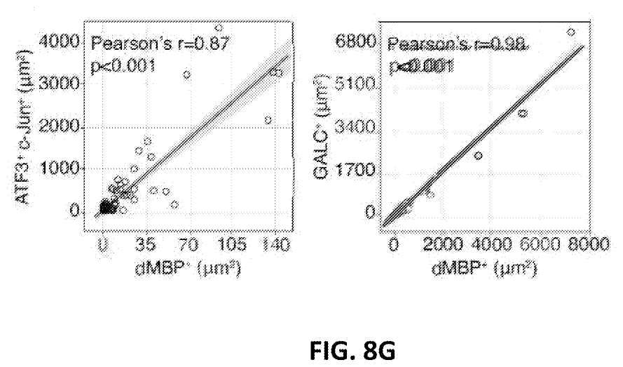

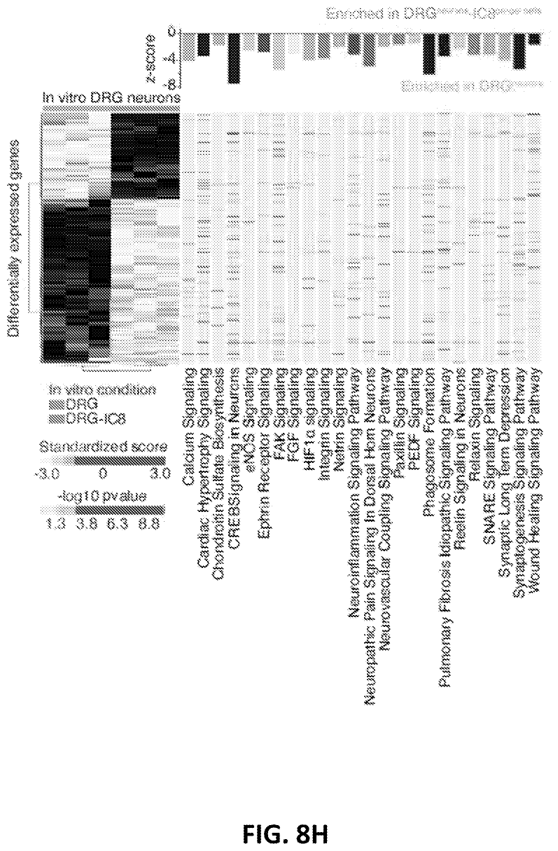

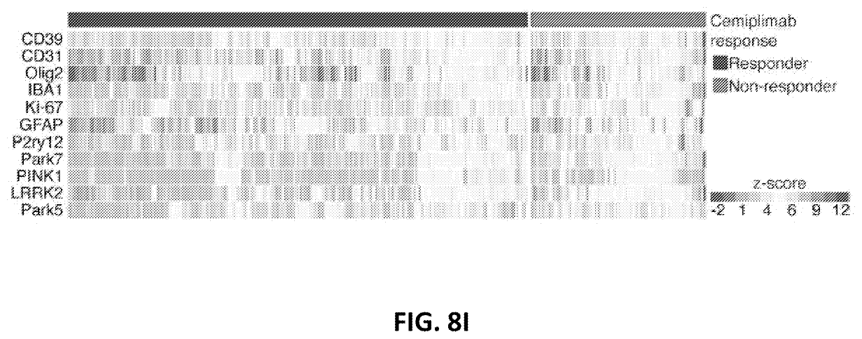

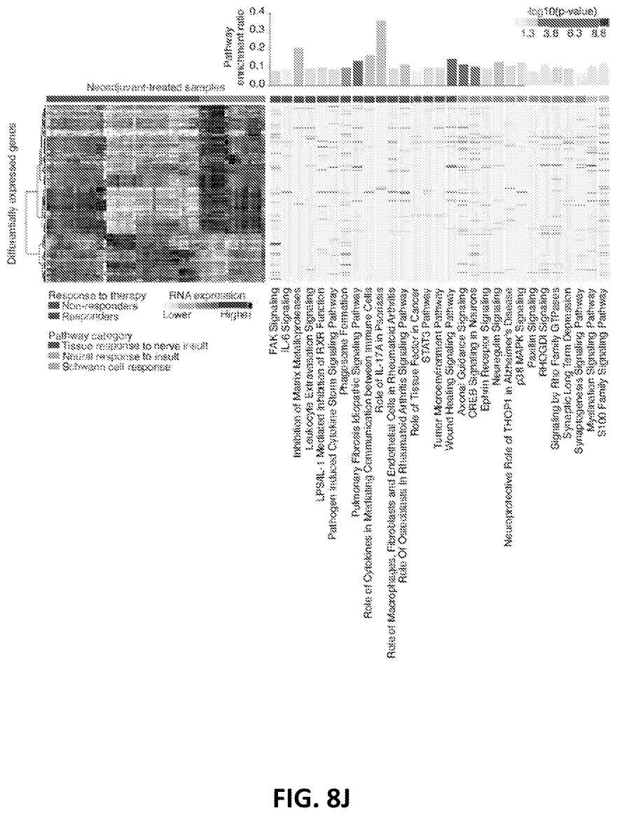

To better understand the mechanism of TAN damage, the interaction between SCC cells and neurons in vitro was examined. Freshly harvested murine dorsal root ganglia (DRG) neurons were kept intact to maintain the integrity of the explant and prevent compromise of the cell-cell contact between neurons, Schwan cells, and endoneurial macrophages. DRG neurons were co-cultured with murine SCC cells (Moc1 and B6). The ultra-structural changes associated with the direct cancer-neuron contact were assessed using electron microscopy (EM). Scanning EM images were obtained on day 5 of the co-culture and confirmed the cancer cell attachment and invasion to the epineurium (FIG. 8A). Compared to naïve neurons, co-cultured neurons demonstrated impaired myelin integrity. The myelin debris presented as large circular aggregates, which were distinct from the linear appearance of normal compact myelin (FIG. 8A). On transmission EM, disintegration was evident at the endoneurial and axonal level, with disentanglement of the compact myelin lamellae (FIG. 8B). Following seven days of co-culture, a complete loss of the myelin sheath was observed at the point of contact with cancer cells, as well as proximal loss of compact myelin integrity and axonal mitochondria-implying the presence of retrograde Wallerian degeneration (FIG. 8C).

Next, high-throughput electrical conduction studies, which utilized murine cSCC model, were performed to show in vivo evidence of cancer induced myelin degradation. Using multi-microelectrode array (MEA), electrophysiological recordings were obtained from 3-5 mm orthotopic cSCC (intradermally injected B6 cells) and from non-tumor bearing healthy control skin (FIG. 8D).

Simultaneous spatial and temporal continuous recording of the extracellular field potential (FP) revealed that, at baseline, spontaneous potentials of tumor bearing skin was comparable with normal skin. However, upon stimulation, the evoked electrical activity in tumor bearing skin was significantly lower than the activity in normal skin (41.8 μV and 60.2 μV, respectively, p<0.0001, FIG. 8D) with significantly increased thresholds. Reversion (“back to baseline”) potentials were comparable (FIG. 8E), but tumor bearing skin demonstrated compound potentials, suggestive of demyelination.

As support that the mechanism driving cancer induced nerve damage was de-myelination, multiplex immunofluorescence stains were conducted on tumor samples from an independent validation cohort of 86 treatment naïve cSCC patients. This cohort included patients with localized (T1-350) disease who underwent Mohs surgery. This external cohort was used to test the hypothesis that cancer induced nerve damage occurs early in the disease course and represent an intrinsic cancer cell trait, rather than a marker of advanced disease.

Tissue sections were stained for general nerve markers (beta-3-tubulin “B3T”), markers of nerve damage (cJUN and ATF3), and markers of de-myelination (degraded myelin base protein, dMBP, and galactosylceramidase (GALC), (FIG. 8F). A significant correlation between nerve insult (ATF3+cJUN+) and de-myelination was observed (dMBP+, pearson's correlation co-efficient=0.87 p<0.0001, see FIG. 8G).

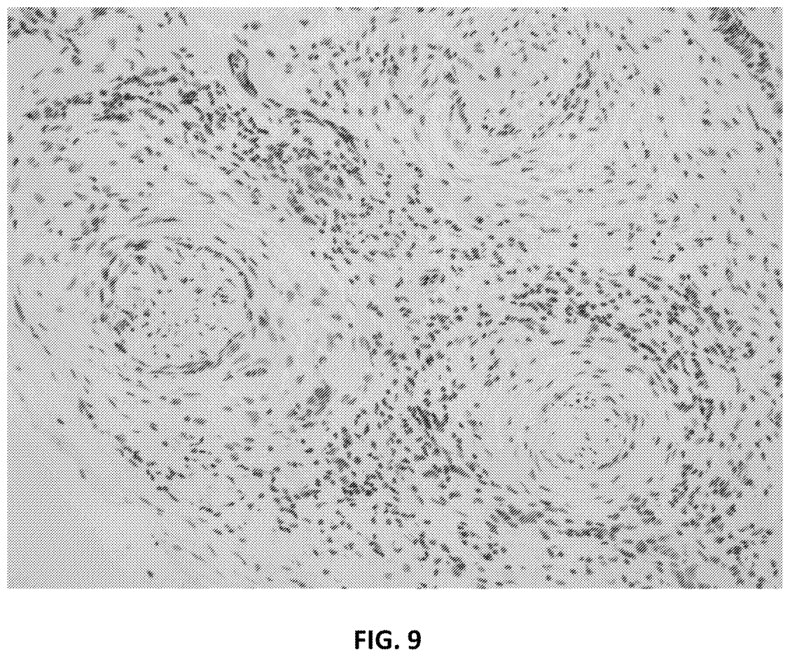

However, due to the proximity of blood vessels to nerve in the TME (neurovascular bundles), potential causes for vascular injury that might contribute to the nerve damage were assessed. Immunohistochemistry stain against ERG, a marker for endothelial cells, revealed that nerve damage was not associated with a vascular injury (FIG. 9A). These findings further confirmed that TAN damage was associated with peripheral demyelination.

As demyelination is a hallmark of central neurodegenerative diseases, such as Parkinson's disease, Alzheimer's disease, and amyotrophic lateral sclerosis, further whether transcriptomic pathways associated with these central neurodegenerative diseases might be present in peripheral nerves exposed to cancer.

Freshly harvested human DRG neurons were co-cultured with human cSCC cells (IC845) for 5 days. The cells were sorted, and NeuO+ cells (live neurons) underwent RNA sequencing. Compared to a neuron-only controls, neurons that were co-cultured with cancer cells significantly downregulated genes involved in homeostasis, neuronal repair, and neuronal survival pathways, including the CREB pathway, FAK signaling, synaptogenesis, phagosome formation, calcium signaling, and SNARE complex, FDR<0.01, see FIG. 8H). To assess potential direct effect of anti-PD-1, human DRG neurons were co-cultured with cSCC cells with and without anti-PD-1 antibodies (Cemiplimab).

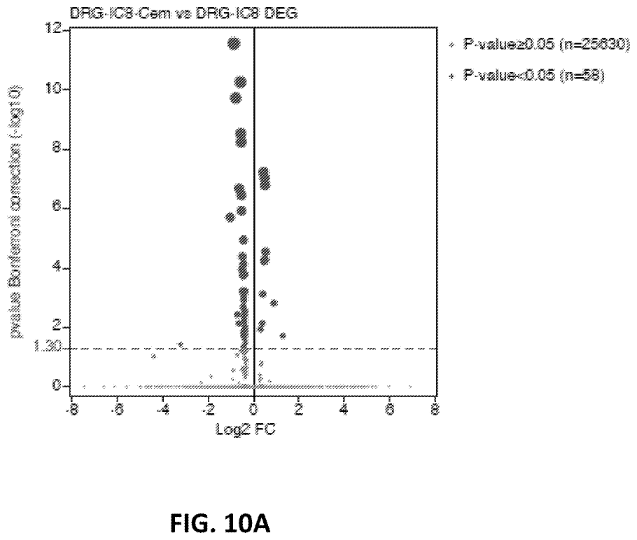

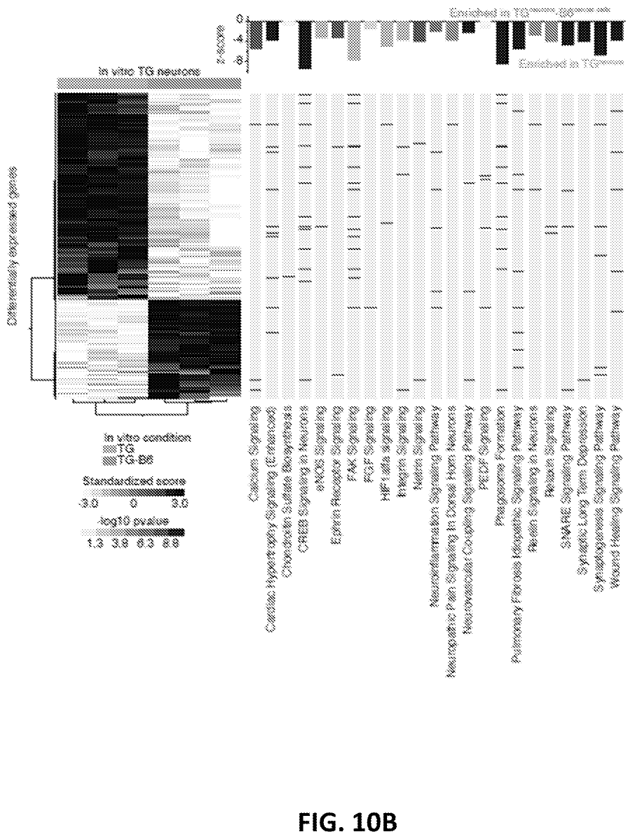

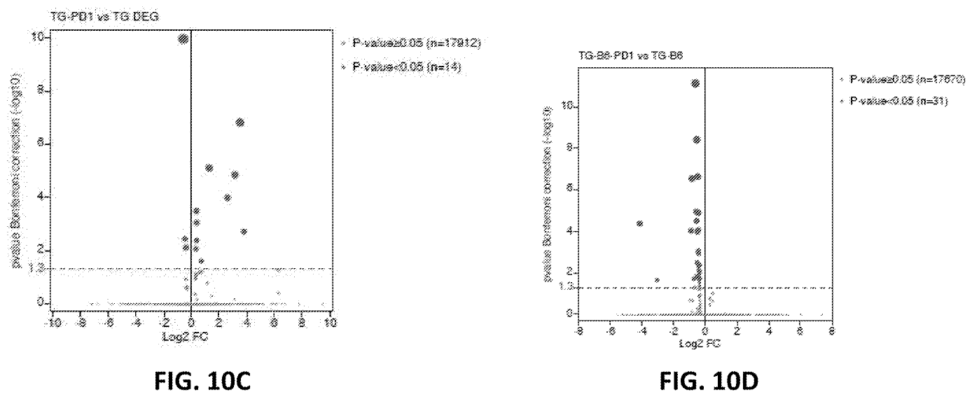

DRG neurons co-cultured with cancer and anti-PD-1 were observed to have similar transcriptional profile compared to DRG neurons co-cultured with cancer without anti-PD1 (58 differentially expressed genes out of 25,688 overall identified genes, 0.2%, see FIG. 10A).

Since tumors in the head and neck region are innervated by the trigeminal ganglia (TG), this experiment was repeated using murine TG neurons co-cultured with murine cSCC B6 cells. The results for the murine TG model were similar to those of the human DRG model—i.e., downregulation of multiple canonical nerve homeostatic pathways, including neuronal repair, synaptogenesis and neuroinflammatory pathways in TG neurons co-cultured with cancer cells compared to TG alone (FIG. 10B-FIG. 10D). Overall, these findings suggested that direct interaction between cancer cells and neurons result in cancer associated peripheral nerve degeneration (CAPND).

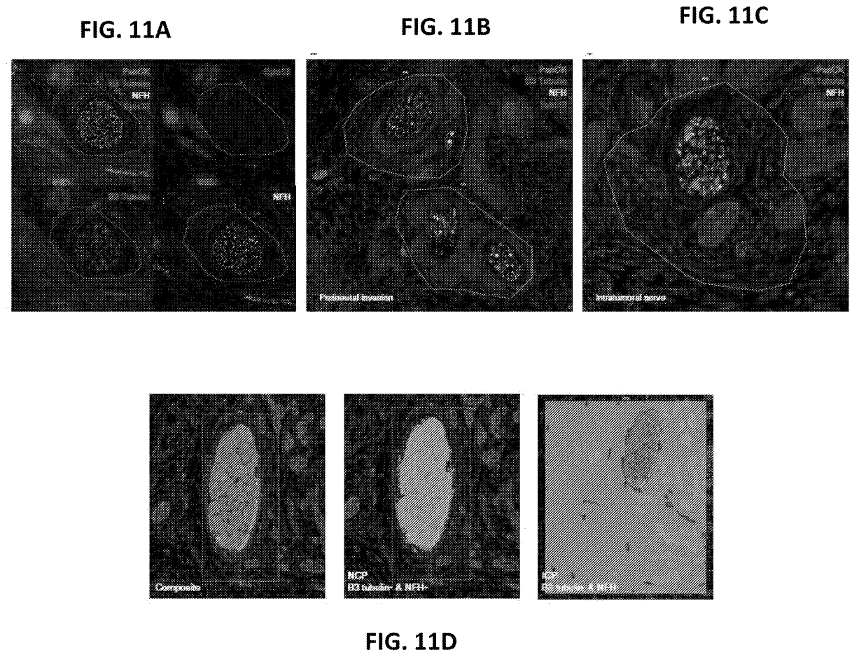

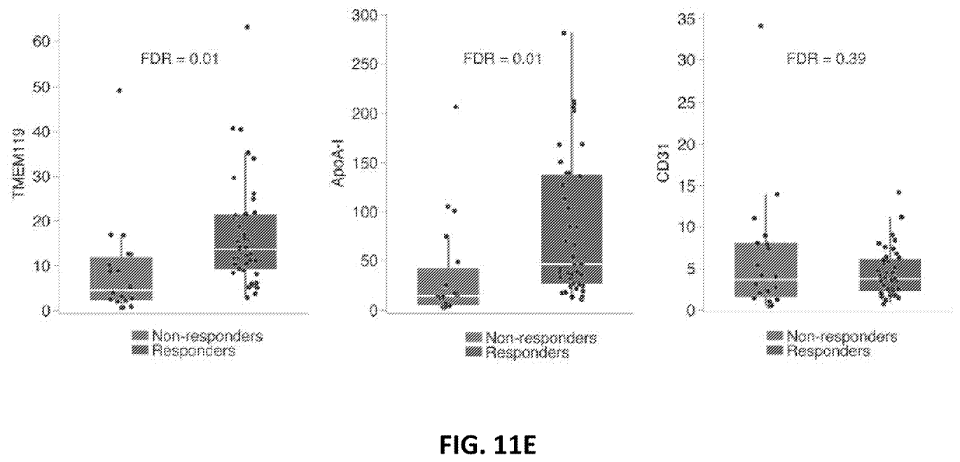

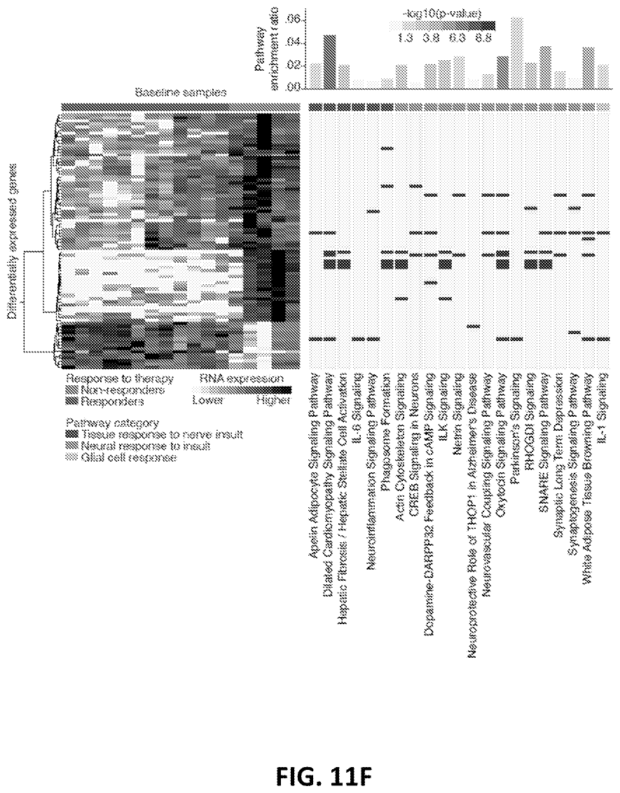

Next, evidence of CAPND in the human cSCC clinical trials cohort was assessed. The degeneration-regeneration homeostatic status of TANs was studied via NanoString GeoMx Digital Spatial Profiler (DSP). The protein neuron profiling panels included markers of neural degeneration (e.g., α-synuclein, LRRK2 and Park5/7) and neuro-inflammation (e.g., IBA1 and TMEM119). Neural niches were identified via immune labeling of the nerve morphology markers neurofilament heavy chain (NFH) and B3T. A total of 553 neural niches (region of interests, ROI, n=84 for baseline samples, n=469 for neoadjuvant-treated) were identified and analyzed using the nCounter protein expression analysis system (FIG. 11A-FIG. 11D). The expression of neurodegenerative proteins in neoadjuvant-treated samples of non-responders (ROI n=109) was compared to those of responders (ROI n=360). Tumors from non-responding patients exhibited increased protein expression of PARK7 (FDR<0.0001), PARK5 (FDR=0.01), PINK1 (FDR=0.006), and LRRK2 (FDR<0.0001), as well as activation and proliferation markers of Schwann cells such as P2RY12 (FDR=0.001), OLIG2 (FDR=0.01), and GFAP (FDR=0.009) see FIG. 8A.

Further analysis of the baseline tumor samples exhibited the neuro-protective protein APOA-I (FDR=0.01) and the neuronal repair microglia marker TMEM119 (FDR=0.01), were over-expressed among responders versus non-responders (FIG. 11E). This DSP protein data was validated by bulk tumor RNA sequencing analysis. Ingenuity Pathway Analysis (IPA) of neoadjuvant-treated samples further exhibited that non-responders significantly up-regulated pathways associated with neural response to injury, such as CREB signaling in neurons, FAK signaling, synaptic excitability, phagosome formation, and myelination neuroprotective role of THOP1 in Alzheimer's disease (FDR<0.01, see FIG. 8J).

Analysis of baseline samples showed that compared to responders, non-responders exhibited significantly downregulated LXR/RXR, RHOGDI signaling. Non-responders exhibited downregulated antioxidant action of ascorbic acid-needed for proper nerve recovery and a conclusion of the repair processes following nerve damage (all FDR<0.01, see FIG. 11F).

Overall, these findings suggested that non-responders to anti-PD-1 therapy had a higher degree of cancer associated peripheral nerve degeneration in both their neoadjuvant-treated and baseline samples. Cancer associated peripheral nerve degeneration correlated with inflammation and tumor promoting TME.

### Example 8. Cancer Associated Peripheral Nerve Degeneration (CAPND) Correlates With an Inflammatory and Tumor Promoting TME

To assess the correlation between cancer associated peripheral nerve degeneration (CAPND) and poor clinical outcome, the expression of neurodegeneration-related gene pathways in SCC samples was assessed from the Cancer Genome Atlas (TCGA). As a cSCC patient cohort was not available at the TCGA database, a cohort of mucosal head and neck SCC (HNSCC) was assessed. HNSCC is a different cancer type than cSCC, therefore this TCGA analysis served as an external validation. The TCGA analysis focused on HNSCC patients with stage II-IVa (n=462), as these were the stages of the clinical trial cSCC patient cohorts.

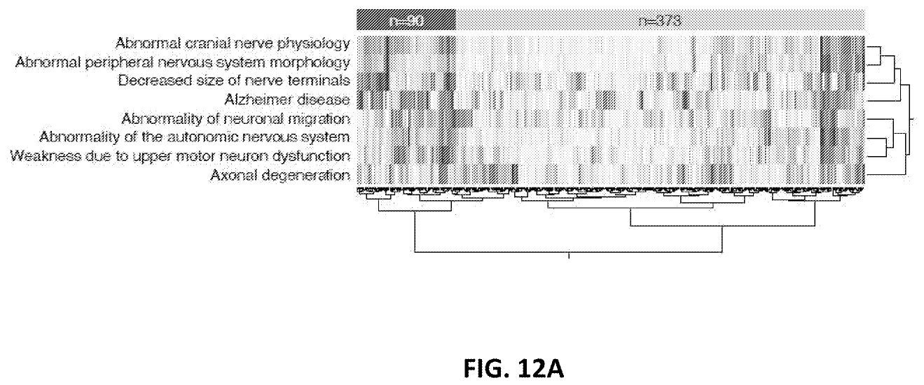

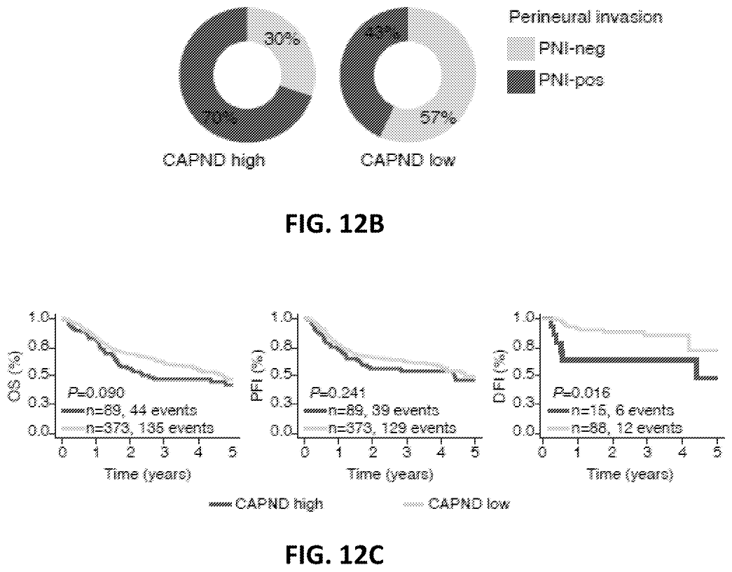

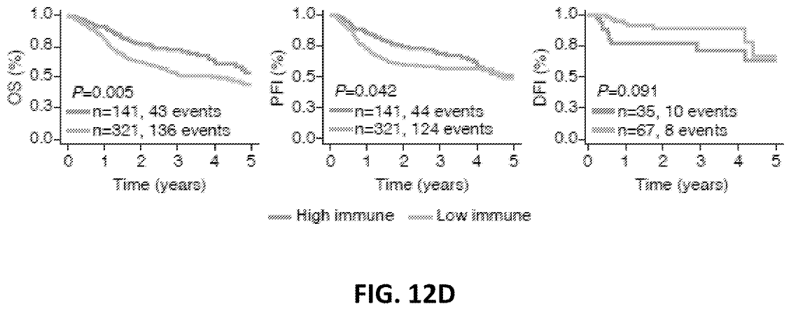

A CAPND signature was created based on neurodegeneration pathways that were enriched in the cSCC clinical trial cohorts (e.g., Alzheimer disease, Human Phenotype Ontology [HPO] M35868 axonal degeneration, HPO M38571; see FIG. 12A). A CAPND enrichment score was calculated for each patient, indicating enrichment for neurodegenerative pathways. Of note, CAPND scores correlated with the presence of PNI (p=0.0011, see FIG. 12B) but not with disease site, stage and HPV status. Among the TCGA HNSCC patients, 90 had a high CAPND score, while 373 patients had a low CAPND score. In comparison with low CAPND score patients, the high score patient cohort displayed a shorter disease-free interval (P=0.016), and an overall worse trend toward overall survival (P=0.09, see FIG. 12C).

The TCGA HNSCC cohort was then stratified into two groups based on an anti-tumoral immune signature, which was previously associated with clinical response to anti-PD-1 therapy in HNSCC patients (data not shown). Kaplan-Meier estimates of overall survival (OS), progression-free interval (PFI), and disease-free interval (DFI) of the TCGA HNSCC patients stratified by the immune response groups (all p values for survival rate differences were calculated using Log-rank analysis, see FIG. 12D).

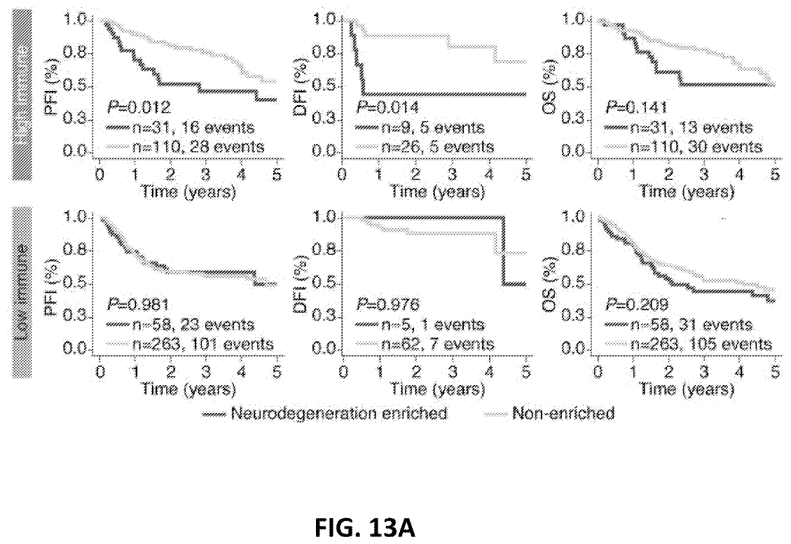

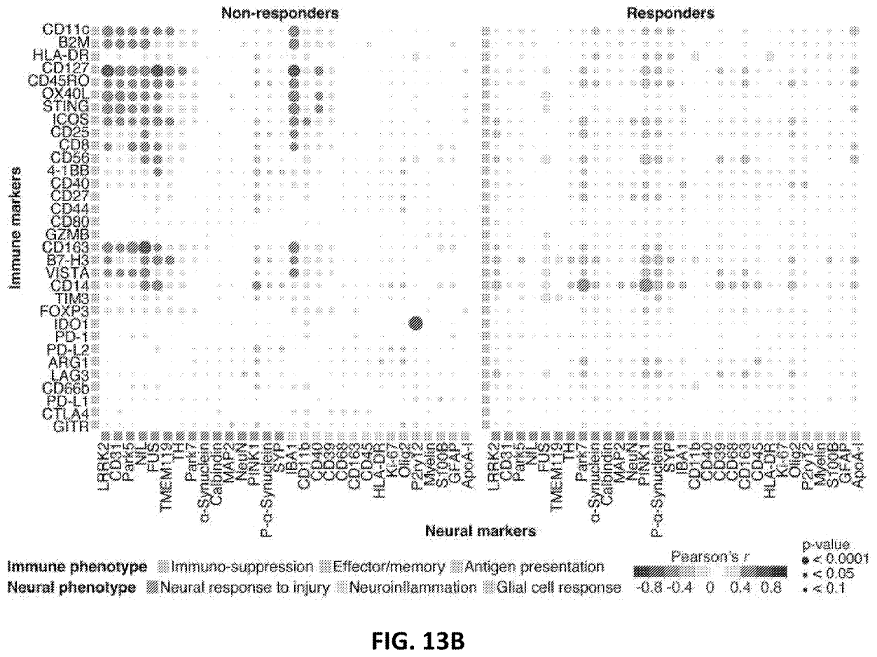

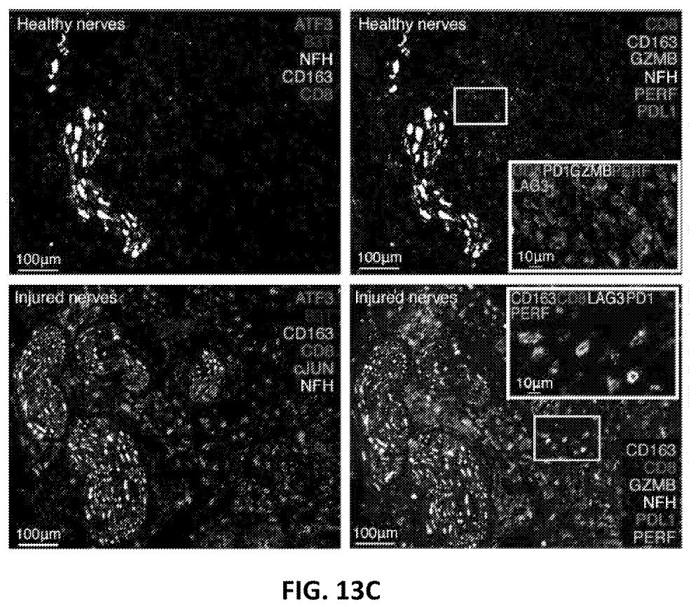

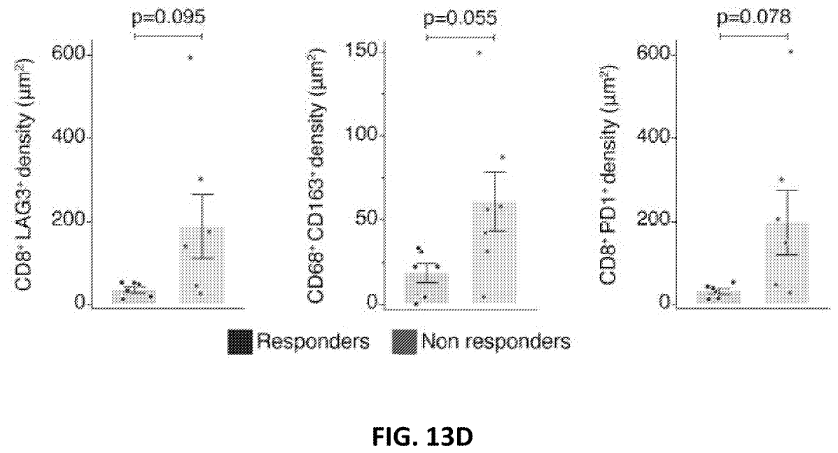

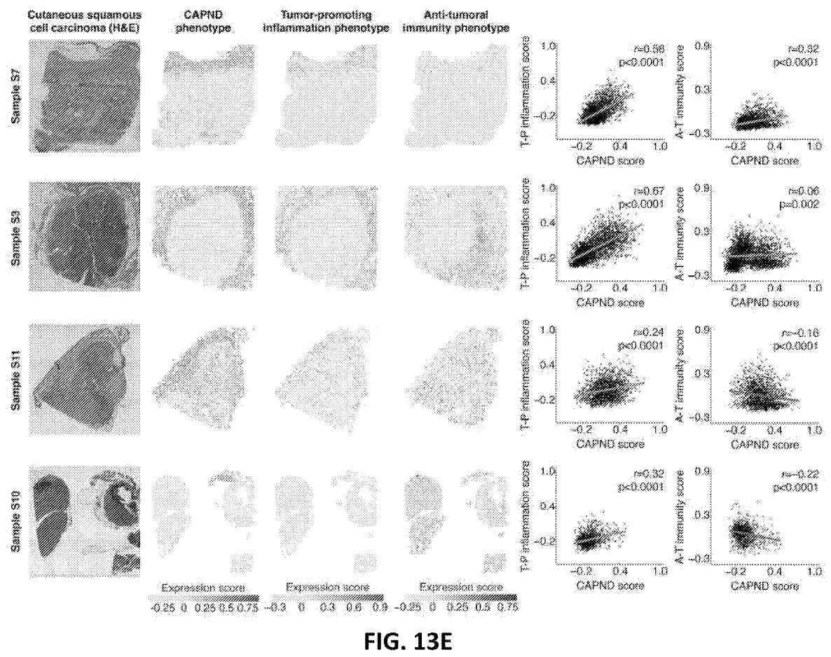

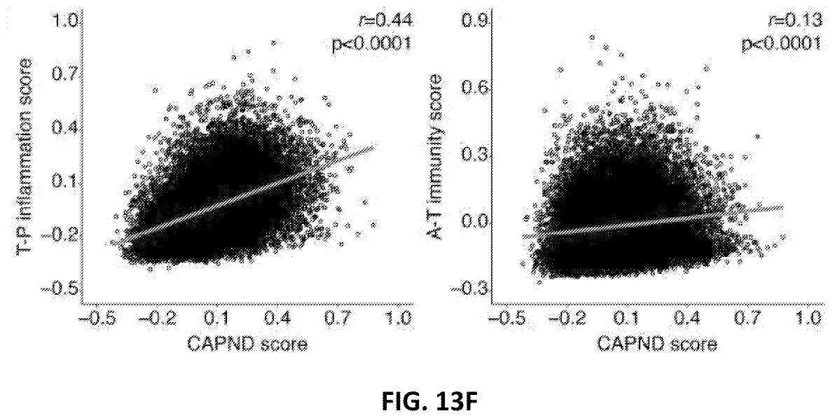

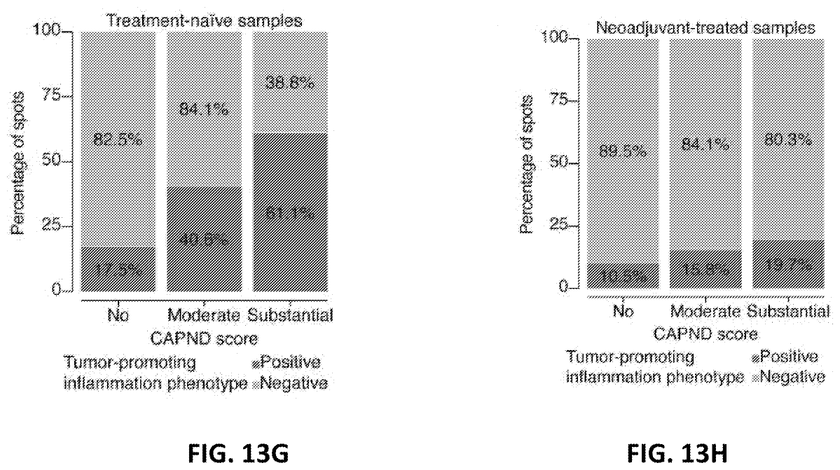

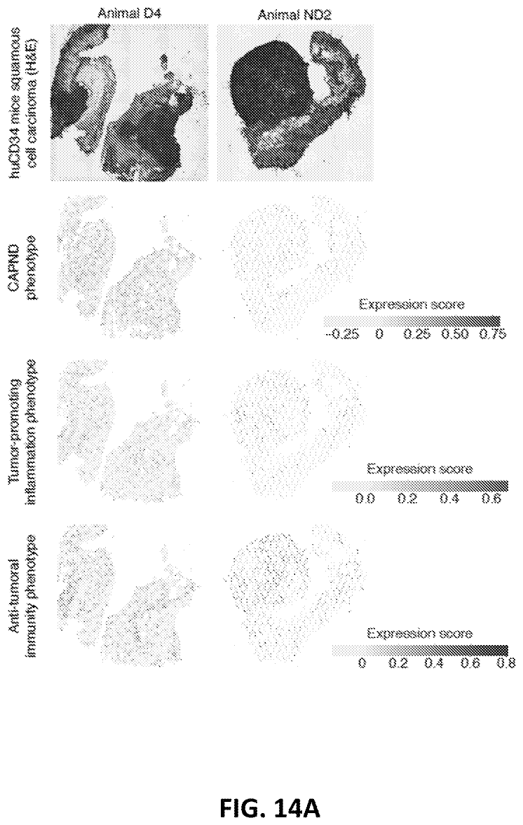

Among patients with high anti-tumoral immunity score (FIG. 13A), a high CAPND score was associated with worse disease-free survival (P=0.014) and progression free survival (P=0.012), but not with overall survival. This correlation of CAPND with adverse disease free and progression free survival remained statistically significant after adjustments for clinical variables such as age, HPV status, site, and staging.

CAPND score did not correlate with survival in patients with low anti-tumoral immunity. These TCGA findings suggested that CAPND can impair the activity of an existing intra-tumoral immune infiltration.

Following a peripheral nerve injury, neurons and Schwan cells attract immune cells to the peri-neural niche to initiate an inflammatory response aimed at nerve healing and regeneration. Hence, CAPND was associated with the presence of pro-inflammatory, tumor promoting immune activity. The potential differences in the peri-neural niche immune activity was then assessed between responders and non-responders. This architectural analysis was done using the DSP protein expression data. Peri-neural niches of neoadjuvant-treated non-responders showed correlation between markers of neuronal response to injury and various immune markers, including immune makers associated with tumor progression such as CD163 (tumor associated macrophages), FOXP3 (T regulatory cells, Tregs), and the immune checkpoints VISTA and IDO-1 (FIG. 13B). In contrast, peri-neural niches of responders showed mainly an inverse correlation between markers of neuronal response to injury and immune markers. These findings were validated using multiplex immunofluorescence stains of the peri-neural niches (FIG. 13C). Analysis of the peri-neural niches (defined as an area within 150 nm from the epicenter of TANs59) in neoadjuvant-treated samples showed that CD68+CD163+ cells, as well as CD8+PD1+ and CD8+LAG3+ cells (exhausted CD8+ T cells) were abundant in non-responders compared to responders (p=0.055, p=0.078, and p=0.095, respectively) see FIG. 13D. Collectively, these findings indicated co-localization of a CAPND and inflammatory, tumor promoting immune activity.

To assess the impact of CAPND on the global anti-tumoral immune activity, spatial transcriptomic analysis on tumor samples from independent, treatment-naïve cSCC patients who underwent surgery (n=11 patients), were performed. Within this spatial analysis, co-localization status of three functional phenotypes was reviewed:
CAPND (expressing the nerve markers
NEFL+NEFH+NEFM+NEUROD1+MRGPRD+TAC1+SSTR2+HAPLN4+SST+; and positive for nerve damage markers ATF3, JUN, SOX1, SMAD1, BHLHE41, KLF7, or KLF6), anti-tumoral immunity (CD8A+GZMB+PRF1+ and CD4+IL2+ T cells; and CD86+IRF8+TNF+ and CD68+PSMB10+HLADQA1+HLADRA+HLADRB1+ antigen presenting cells), and tumor-promoting inflammation (CD204+CD206+CD163+ and CD68+IL-10+ tumor-associated macrophages; and CD4+FOXP3+ T regulatory cells).

FIG. 13E shows the spatial distribution of the CAPND, tumor-promoting inflammation, and anti-tumoral immunity phenotypes in individual tissue sections and the respective correlation between the CAPND and immunity scores. In this treatment-naïve cohort, the CAPND phenotype correlated with the tumor-promoting inflammation phenotype (R=0.44) but not with the anti-tumoral immunity (R=0.13), see FIG. 13F.

Analysis of 27,420 tumor regions revealed that 61.1% of the regions with substantial presence of the CAPND phenotype (defined as fold change >2 with FDR<0.01) co-localized with the tumor-promoting inflammation phenotype (n=1544 of 8812 and 4780 of 7817, p<0.001, see FIG. 13G).

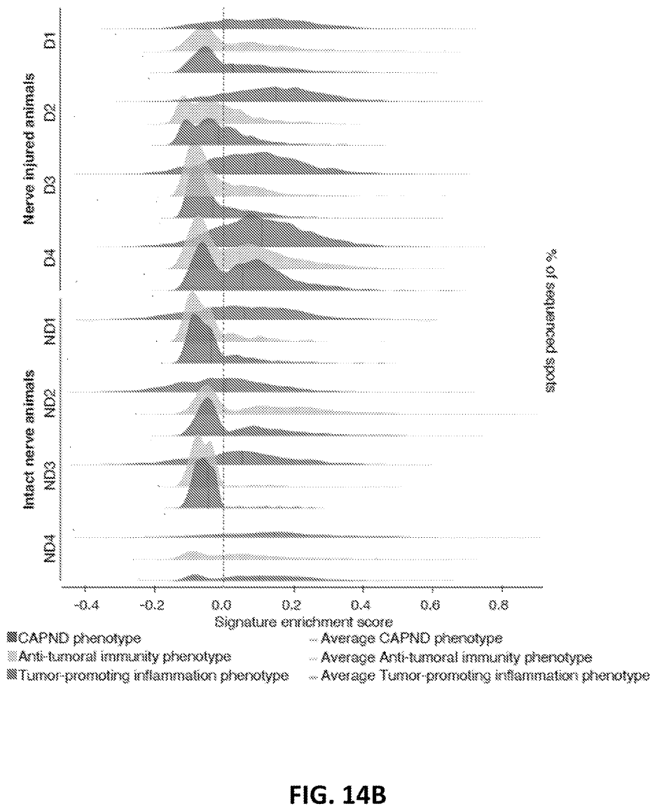

Next, these spatial findings in the cSCC clinical trial cohort were evaluated. Among the neoadjuvant-treated tumors, region with CAPND phenotype co-localized the tumor-promoting inflammation phenotype higher compared to regions without CAPND (n=688 of 6571 and 596 of 3019, p<0.001 FIG. 13H). A similar spatial transcriptomic analysis was conducted on tumors derived from the nerve injury mouse hSCC model (see above) treated with the Cemiplimab. The CAPND phenotype was enriched among axotomized mice compared with sham operated mice (FIG. 14A). These enriched regions were spatially associated with increased tumor-promoting inflammatory activity in axotomized mice compared to sham operated mice, but not with the anti-tumoral immunity phenotype (FIG. 14B).

Taken together, these results suggested a functional role for CAPND in facilitating an inflammatory, tumor-promoting immune activity that affect the general TME immune tone and therefore dampen the clinical efficacy of anti-PD-1 therapy.

### Example 9. Blockade of TAN-Induced Inflammatory Signals Enhanced Anti-PD-1 Efficacy

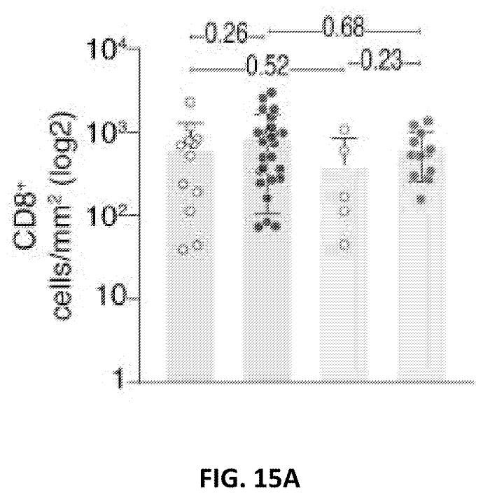

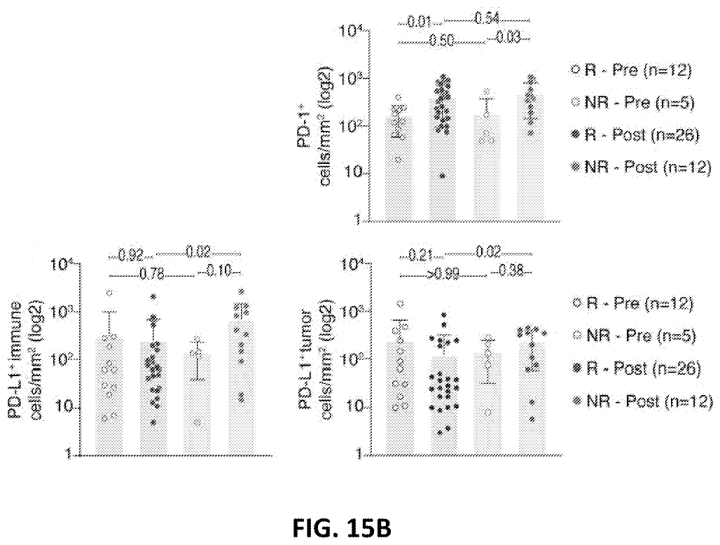

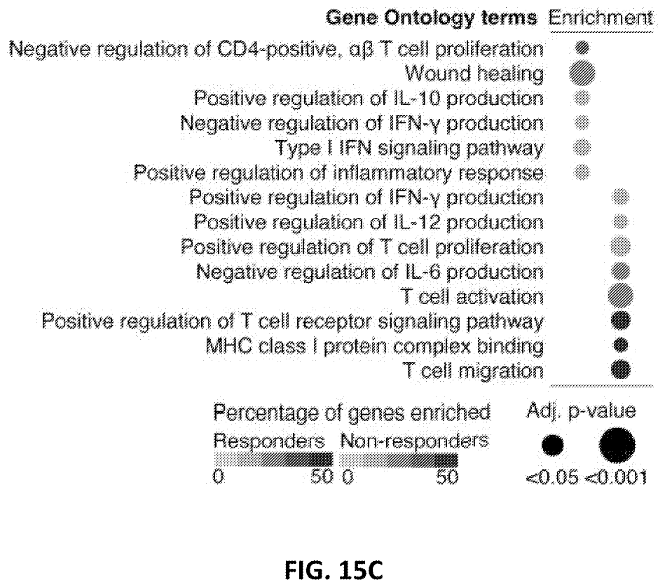

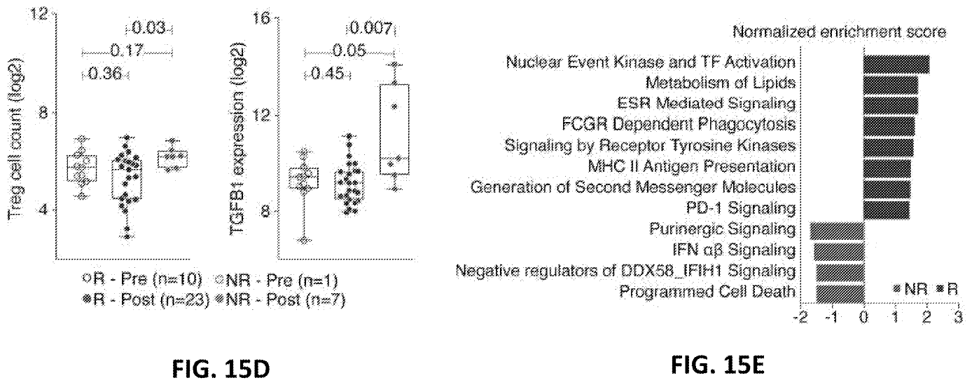

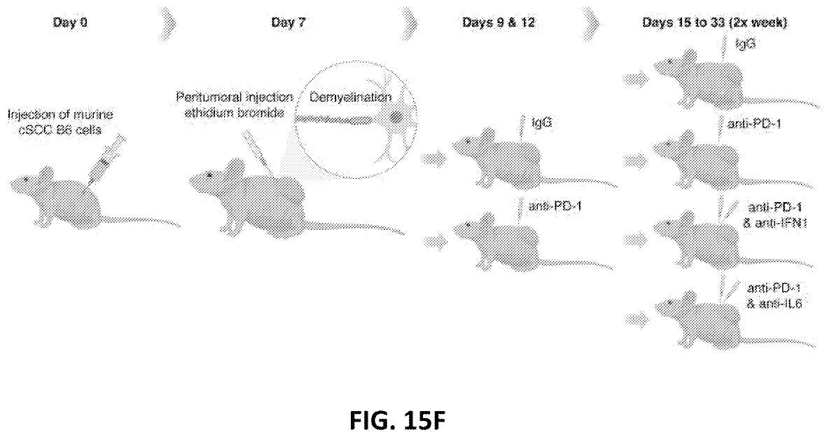

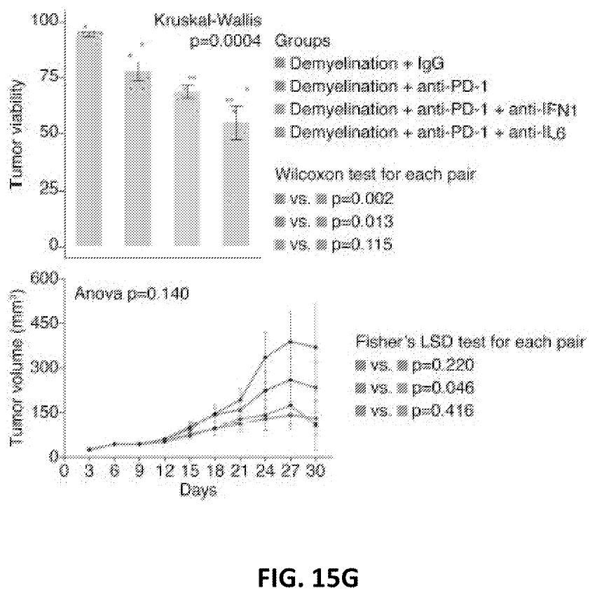

The expansion of pro-nerve healing, tumor-promoting inflammation from the per-neural niche to the rest of the TME was further assessed, intra-tumoral immune difference between responders and non-responders was profiled from the clinical trials cohort. Immunohistochemical staining of tumor samples demonstrated no differences in CD8+ T-cell abundance between responders and non-responders either before or after treatment (FIG. 15A).

CD8+ T-cells could properly infiltrate tumors of non-responding patients t was whether these T-cells encountered a hostile TME, leading to their functional impairment.

To test this hypothesis, PDL-1 was stained, as PDL-1 acts as a negative feedback loop suppressing CD8+ T-cell activation. It was found that neoadjuvant-treated tumors of non-responders had significantly higher expression of PD-L1 on both tumor cells and immune cells compared to responders (p=0.02 and p=0.02, respectively, see FIG. 15B). Next, gene pathway analysis was performed on bulk tumor RNA to assess for functional immune differences. GO pathway analysis of baseline tumor samples did not identify any immune-related pathways that significantly differed according to response status. GO pathway analysis of neoadjuvant-treated tumor of responders demonstrated upregulation of antitumoral immune-related pathways (FIG. 15C), such as upregulation of IL-12 production (GO:0032735; FDR<0.001), T-cell activation (GO:0042110; FDR=0.001), and positive regulation of IFN-α production (GO:0032729; FDR=0.03). In contrast, non-responders upregulated tumor-promoting pathways, such as negative regulation of IFN-β production (GO:0032689; FDR<0.01), positive regulation of IL-10 production (GO:0032733; FDR=0.03), and wound healing (GO:0042060; FDR<0.01).

Type I interferon signaling pathway (GO:0060337) was up-regulated among neoadjuvant treated non-responders (FIG. 15C). This observation was confirmed via NanoString nCounter PanCancer Immune Profiling Panel. The Nanostring analysis demonstrated a higher Treg infiltration and a higher tumor growth factor (TGF)-β-1 expression among neoadjuvant-treated non-responders (FIG. 15D). It also demonstrated upregulation of IFN-α and IFN-β (type I IFN, IFN-I) signaling in the tumors of neoadjuvant-treated non-responders (FIG. 15E). These findings initially seemed counterintuitive, as IFN-I signaling has been associated with a favorable response to immunotherapy. However, while acute IFN-I signaling can promote antitumoral immunity, these results support that chronic IFN-I signaling can drive immunotherapy resistance.

To determine if CAPND can be the source of IFN-I signaling, the in vitro model of DRG neurons cultured alone, or with cSCC cells for 5 days, was then studied (FIG. 15F). RNA sequencing of the neurons from this model demonstrated that neurons co-culture with cancer cells up-regulated not only neurodegenerative pathways, but also IFN-I signaling pathways (Positive regulation of type I interferon production, GO:0032481; FDR<0.0001, and regulation of type I interferon production, GO:0032479; FDR=0.01). Moreover, cancer exposed neurons also up-regulated inflammatory pathways, such as positive regulation of IL-1β production (GO:0032731; FDR<0.0001), and positive regulation of IL-6 production (GO:0032755; FDR<0.0001).

To confirm that CAPND was the source of chronic IFN-I activity among non-responders, non-CAPND related, potential sources for activation of IFN-I signaling were studied. As the microbiome has been previously associated with IFN-I signaling, the tumor-associated microbiome (including analysis of the bacteriome, virome, and mycobiome [fungi]) was analyzed in the cSCC clinical trial samples. No differences in the intra-tumoral microbiome composition between responders and non-responders was observed.

One week after injection of B6 cSCC cells to 36 mice, a demyelinating agent (ethidium-bromide) was injected to the tumor periphery at the tumor-normal skin interface, to reverse the CAPND-derived resistance to anti-PD-1 therapy via blockade of inflammatory signals in vivo (FIG. 15F).

Anti-PD-1 treatment was initiated two days after the demyelination. To allow priming of immune cells against the tumor, the first two anti-PD-1 doses were administered alone. Subsequent anti-PD-1 doses were given alone, or in combination with antibodies blocking either interferon-a-receptor-1 or IL-6. The addition of anti-inflammatory antibodies did not reduce the tumor size in a statistically significant manner (FIG. 15G). However, in both of the neoadjuvant Cemiplimab clinical trials, there was no correlation between the tumor size per imaging to pathological response to treatment, with 51% pathological CR rate versus only 6% of radiological CR31. The addition of anti-IL-6 to anti-PD-1 in the setting of TANs demyelination had significantly improved treatment efficacy, with an average of only 55% viable tumor cells versus 78% viable tumor cells in the anti-PD-1 monotherapy arm (FIG. 15G).

### Example 10. Tan-Derived Anti-PD-1 Resistance is Clinically Targetable and Reversible

The murine model results demonstrated that combined blockade of PD-1 and the pro-inflammatory cytokine IL-6 improved anti-PD-1 efficacy (FIG. 15A-FIG. 15G).

Moreover, markers of nerve degeneration can serve as future bio-markers to identify patients with lower chances of responding to anti-PD-1. These insights aided in identifying biomarkers and developing therapeutic agents targeting specific features of nerve degeneration or its associated tumor-promoting inflammation. Combined with immunotherapy, such agents can improve patients care across different tumor types.

1. A combination therapy comprising:
(a) an antibody that specifically binds to an immune checkpoint protein; and
(b) an antibody that specifically binds to a neurological biomarker selected from the group consisting of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1(CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription Factor 3 (ATF3), cJUN, NEUROD1, RGPRD, TAC1, SSTR2, HAPLN4, SST, SMAD1, BHLE41, KLF7, KLF6, Calcitonin Gene-Related Peptide (CGRP), Choline Acetyltransferase (ChAT), Vesicular Acetylcholine Transporter (VAChT), and any combination thereof.

2. The combination therapy of claim 1, wherein the antibody specifically binds to the neurological biomarker which is selected from the group consisting of NfH, NfL, TH, GFAP, OLIG2, CD39, ATF3, cJUN, CGRP, ChAT, VAChT, and any combination thereof.

3. The combination therapy of any one of claims 1-2, wherein the neurological biomarker is NfH.

4. The combination therapy of any one of claims 1-2, wherein the neurological biomarker is NfL.

5. The combination therapy of any one of claims 1-2, wherein the neurological biomarker is TH.

6. The combination therapy of any one of claims 1-2, wherein the neurological biomarker is GFAP.

7. The combination therapy of any one of claims 1-2, wherein the neurological biomarker is OLIG2.

8. The combination therapy of any one of claims 1-2, wherein the neurological biomarker is CD39.

9. The combination therapy of any one of claims 1-2, wherein the neurological biomarker is ATF3.

10. The combination therapy of any one of claims 1-2, wherein the neurological biomarker is cJUN.

11. The combination therapy of any one of claims 1-2, wherein the neurological biomarker is CGRP.

12. The combination therapy of any one of claims 1-2, wherein the neurological biomarker is ChAT.

13. The combination therapy of any one of claims 1-2, wherein the neurological biomarker is VAChT.

14. The combination therapy of any one of claims 1-13, wherein the antibody that binds to an immune checkpoint protein is selected from the group consisting of an anti-PD-1 antibody, anti-CTLA-4 antibody, anti-LAG-3 antibody, anti-4-1BB antibody, anti-OX-40 antibody, anti-CD27 antibody, anti-CD28 antibody, anti-TIM3 antibody, anti-B7-H3 antibody, anti-VISTA antibody, anti-IDO-1 antibody, and any combination thereof.

15. The combination therapy of any one of claims 1-14, wherein the antibody that binds to an immune checkpoint protein is an anti-PD-1 antibody.

16. The combination therapy of any one of claims 1-15, wherein the antibody that binds to an immune checkpoint protein is an anti-PD-1 antibody is selected from the group consisting of nivolumab, pembrolizumab, cemiplimab, and dostarlimab.

17. The combination therapy of any one of claims 1-16, wherein the antibody that specifically binds to the immune checkpoint protein and the antibody directed to the neurological biomarker are in the same composition.

18. The combination therapy of any of claims 1-16, wherein the antibody that specifically binds to the immune checkpoint protein and the antibody directed to the neurological biomarker are in separate compositions.

19. The combination therapy of any one of the previous claims wherein, the antibody that specifically binds to the immune checkpoint protein is suitable for intratumorally, intravenously, intraperitoneally, intramuscularly, intrathecally or subcutaneous delivery.

20. The combination therapy of any one of the previous claims, wherein the antibody that specifically binds to the immune checkpoint protein is suitable for intravenous delivery.

21. The combination therapy of any one of the previous claims, wherein the antibody directed to the neurological biomarker is suitable for intratumorally, intravenously, intraperitoneally, intramuscularly, intrathecally or subcutaneous delivery.

22. A method of treating a subject suffering from cancer comprising administering to the subject a therapeutically effective amount of a combination therapy of any of the previous claims to the subject.

23. The method of claim 22, wherein the antibody that specifically binds to the immune checkpoint protein and the antibody directed to the neurological biomarker are administered simultaneously or sequentially.

24. The method of claim 22, wherein the antibody that specifically binds to the immune checkpoint protein and the antibody directed to the neurological biomarker are administered in the same composition.

25. The method of claim 22, wherein the antibody that specifically binds to the immune checkpoint protein and the antibody directed to the neurological biomarker are administered in different compositions.

26. The method of any one of claims 22-25, wherein the antibody that specifically binds to the immune checkpoint protein is administered prior to or at the time of administration of the antibody directed to the neurological biomarker.

27. A method of treating a subject suffering from a cancer comprising:
(a) detecting the presence of or assessing the presence or absence of a neurological biomarker in a biological sample of the subject, wherein the neurological biomarker is selected from the group consisting of Synaptophysin, PTEN-Induced Kinase 1 (PINK1), Leucine Rich Repeat Kinase 2 (LRRK2), Microtubule-Associated Protein 2 (MAP2), Parkinsonism Associated Deglycase (Park7), Phospho-Alpha-Synuclein (S129), Transmembrane Protein 119 (TMEM119), Platelet Endothelial Cell Adhesion Molecule (CD31), Integrin Alpha M (CD11b), Ectonucleoside Triphosphate Diphosphohydrolase-1(CD39), Alpha-Synuclein, Purinergic Receptor P2Y12 (P2ry12), Apolipoprotein (Apoa-I), Ubiquitin Carboxy-Terminal Hydrolase L1 (Park5), Calbindin, FUS, Myelin Basic Protein, Glial Fibrillary Acidic Protein (GFAP), S100 Calcium Binding Protein B (S100B), Neun, Ionized Calcium Binding Adaptor Molecule (IBA1), Oligodendrocyte Transcription Factor 2 (Olig2), Tyrosine Hydroxylase (TH), Neurofilament Light Chain (NfL), Neurofilament Heavy Chain (NfH), Activating Transcription factor 3 (ATF3), cJUN, Calcitonin Gene-Related Peptide (CGRP), Choline Acetyltransferase (ChAT), Vesicular Acetylcholine Transporter (VAChT), and any combination thereof, and
(b) administering to the subject a therapeutically effective amount of (i) an immune checkpoint inhibitor, and/or (ii) an antibody directed to a neurological biomarker selected from any of the neurological biomarkers in (a), if the neurological biomarker is determined to be present in the biological sample of the subject.

28. The method of claim 27, wherein the antibody specifically binds to the neurological biomarker which is selected from the group consisting of NfH, NfL, TH, GFAP, OLIG2, CD39, ATF3, cJUN, CGRP, ChAT, VAChT, and any combination thereof.

29. The method of any one of claims 27-28, wherein the neurological biomarker is NfL.

30. The method of any one of claims 27-28, wherein the neurological biomarker is NfH.

31. The method of any one of claims 27-28, wherein the neurological biomarker is TH.

32. The method of any one of claims 27-28, wherein the neurological biomarker is GFAP.

33. The method of any one of claims 27-28, wherein the neurological biomarker is OLIG2.

34. The method of any one of claims 27-28, wherein the neurological biomarker is CD39.

35. The method of any one of claims 27-28, wherein the neurological biomarker is ATF3.

36. The method of any one of claims 27-28, wherein the neurological biomarker is cJUN.

37. The method of any one of claims 27-28, wherein the neurological biomarker is CGRP.

38. The method of any one of claims 27-28, wherein the neurological biomarker is ChAT.

39. The method of any one of claims 27-28, wherein the neurological biomarker is VAChT.

40. The method of any one of claims 27-39, wherein biological sample is selected from a group consisting of blood, urine, tumor tissue, and cerebrospinal fluid (CSF).

41. The method of any one of claims 27-40, wherein if the subject is identified as exhibiting the absence of, or a low level of a neurological biomarker in the biological sample relative to a reference sample, the subject is not administered (i) an immune checkpoint inhibitor, and/or (ii) an antibody directed to a neurological biomarker selected from any of the neurological biomarkers in (a).

42. The method of any one of claims 27-40, wherein if the subject is identified as exhibiting the presence of or a determined high level of a neurological biomarker in the biological sample relative to a reference sample, the subject is not administered (i) an immune checkpoint inhibitor, and/or (ii) an antibody directed to a neurological biomarker selected from any of the neurological biomarkers in (a).

43. The method of any one of claims 27-42, wherein the immune checkpoint inhibitor is administered intratumorally, intravenously, intraperitoneally, intramuscularly, intrathecally or subcutaneously.

44. The method of any one of claims 27-42, wherein the antibody directed to the neurological biomarker is administered intratumorally, intravenously, intraperitoneally, intramuscularly, intrathecally or subcutaneously.

45. The method of any one of claims 27-44, wherein the immune checkpoint inhibitor and the antibody directed to the neurological biomarker are administered simultaneously or sequentially.

46. The method of any one of claims 27-45, wherein the immune checkpoint inhibitor and the antibody directed to the neurological biomarker are administered in the same composition.

47. The method of any one of claims 27-45, wherein the immune checkpoint inhibitor and the antibody directed to the neurological biomarker are administered in different compositions.

48. The method of any one of claims 27-47, wherein the immune checkpoint inhibitor is administered prior to or at the time of administration of the antibody directed to the neurological biomarker.

49. The method of any one of claims 22 or 48, wherein the administration of the immune checkpoint inhibitor inhibits the recurrence of a lesion.

50. The method of any one of claims 22 or 49, wherein the cancer is selected from the group consisting of breast cancer, bladder cancer, cervical cancer, colon cancer, head and neck cancer, Hodgkin lymphoma, liver cancer, lung cancer, renal cell cancer (a type of kidney cancer), skin cancer (including melanoma, squamous cell carcinoma, or a cutaneous squamous cell carcinoma (cSCC)), stomach cancer, rectal cancer, or any combination thereof.

51. The method of any one of claims 19 or 44, wherein the cancer is selected from the group consisting of advanced cSCC, metastatic cSCC, locally advanced cSCC, resectable cSCC, unresectable cSCC, recurrent cSCC, and any combination thereof.
